(19) United States
(12) Patent Application Publication
(10) Pub. No.: US 2023/0183187 A1
(43) Pub. Date: Jun. 15, 2023

(54) QUINAZOLINE COMPOUNDS AS INHIBITORS OF PREMATURE TERMINATION CODONS

(71) Applicants: CENTRE NATIONAL DE LA RECHERCHE SCIENTIFIQUE, Paris (FR); INSTITUT NATIONAL DE LA SANTE ET DE LA RECHERCHE MEDICALE (INSERM), Paris (FR); COMMISSARIAT À L'ÉNERGIE ATOIMQUE ET AUX ÉNERGIES ALERNATIVES, Paris (FR); UNIVERSITE PARIS-SACLAY, Gif-sur-Yvette (FR); SORBONNE UNIVERSITÉ, Paris (FR); ASSOCIATION FRANÇAISE CONTRE LES MYOPATHIES, Paris Cedex 13 (FR); ASSOCIATION INSTITUT DE MYOLOGIE, Paris (FR)

(72) Inventors: Olivier NAMY, Saclay (FR); Laure BIDOU, Paris (FR); Olivier BUGARD, Le Plessis Robinson (FR); Jean-Christophe CINTRAT, Igny (FR); Goulven MERER, Versailles (FR); Egor CHIRKIN, Palaiseau (FR)

(21) Appl. No.: 17/998,436

(22) PCT Filed: May 12, 2021
(86) PCT No.: PCT/EP2021/062635

§ 371 Date: Nov. 10, 2022

(30) Foreign Application Priority Data
May 12, 2020 (EP) 20305478.8

(51) Int. Cl. C07D 239/84

(52) U.S. Cl. CPC C07D 239/84

(57) ABSTRACT

The present invention relates to the use of at least one compound of formula (I), or one of its pharmaceutically acceptable salts, for preventing and/or treating a disease caused by a nonsense mutation. It also relates to compounds of formula (II) and their uses.

The present invention relates to the use of at least one compound of formula (I), or one of its pharmaceutically acceptable salts, for preventing and/or treating a disease caused by a nonsense mutation. It also relates to compounds of formula (II) and their uses.

The treatment of diseases involving genetic aspects, particularly such as nonsense mutations, is a challenge of interest.

For eukaryotic organisms such as humans using a standard genetic code, translation termination occurs when one of the three stop codons, UAA, UGA and UAG, enters the ribosomal A-site which is then recognized by the release factor (eRF1). Although accurate, translation termination is not 100% effective and its efficiency depends on competition between the recognition of the stop codon by eRF1, and the decoding of the stop codon by a near-cognate tRNA (i.e. a natural suppressor tRNA). The latter case leads to the suppression of translation termination, also called “readthrough”, where an amino acid is incorporated in place of the stop.

When a nonsense mutation appears in a coding sequence, this creates a premature termination codon (PTC), which prevents the correct production of the corresponding protein, by interrupting translation and inducing degradation of the transcript via the nonsense-mediated mRNA decay (NMD) pathway. Therefore, PTCs are associated with a large number of genetic diseases and cancers. A meta-analysis of nonsense mutations causing human genetic disease highlighted that 11% of inherited diseases arises from PTC mutations, 80% of them being TGA and TAG (Mort et al, A meta-analysis of nonsense mutations causing human genetic disease, Hum Mutat, 2008, August; 29(8):1037-47).

In the last decade, considerable interest has focused on in-frame PTCs as potential therapeutic targets.

Indeed, aminoglycosides (such as paromomycin, gentamicin, G418 also called geneticin, or amikacin) and their derivatives (NB series) have been shown to promote PTC readthrough by binding to mammalian ribosomes and partly restoring the synthesis of a full-length protein in cultured mammalian cells and animal models. The potential of this approach was first demonstrated in vivo by Barton-Davis and coworkers, who reported the restoration of dystrophin levels to 10-20% those in wild-type animals in the skeletal muscle of mdx mice, following subcutaneous injections of gentamicin (Barton-Davis et al, 1999, Aminoglycoside antibiotics restore dystrophin function to skeletal muscles of mdx mice. J Clin Invest, 104, 375-381). This strategy has been evaluated in a large number of genetic diseases (Lee et al, Pharmaceutical therapies to recode nonsense mutations in inherited diseases, Pharmacol Ther 2012 November; 136(2):227-66), including cystic fibrosis (CF), muscular dystrophies (Linde et al, 2008, Introducing sense into nonsense in treatments of human genetic diseases. Trends Genet, 24, 552-563) and mucopolysaccharidosis type I-Hurler (MPS I-H) (Gunn et al, Mol Genet Metab 2014 March; 111(3):374-381, Long-term nonsense suppression therapy moderates MPS I-H disease progression). Several clinical trials, with various rates of success, have already been performed (Bordeira-Carrico et al, 2012, Cancer syndromes and therapy by stop-codon readthrough. Trends Mol Med, 18, 667-678; Keeling et al, 2014, Therapeutics based on stop codon readthrough. Annual review of genomics and human genetics, 15, 371-394). Encouraging results have been obtained in some cases, particularly for mutations displaying high levels of readthrough in the presence of gentamicin (Sermet-Gaudelus, I., Renouil, M., Fajac, A., Bidou, L., Parbaille, B., Pierrot, S., Davy, N., Bismuth, E., Reinert, P., Lenoir, G. et al. (2007) In vitro prediction of stop-codon suppression by intravenous gentamicin in patients with cystic fibrosis: a pilot study. BMC medicine, 5, 5). Despite their therapeutic potential, treatment with aminoglycosides is associated with severe adverse effects, notably with ototoxicity and/or nephrotoxicity.

Thus, there is a need for the development of novel PTC readthrough promoters with reduced toxicity.

Compounds structurally distinct from aminoglycosides have been developed, such as ataluren, negamycin, clitocine or escin.

Ataluren, also known as PTC124, was initially considered highly promising. Despite its clinical benefit still being debated, it recently obtained a conditional approval from EMA. Negamycin is a dipeptide binding the ribosomal A-site that shows a readthough context dependency different from gentamicin. Clitocine is a nucleoside analogue which has demonstrated PTC readthrough activity, although its mechanism of action remains unknown. Escin, a natural mixture of triterpenoid saponins isolated from horse chestnut (Aesculus hippocastanum) seeds, has been recently shown to promote readthrough of G542X and W1282X mutations found in CFTR gene.

However, all of these compounds are specific of a defined PTC, thereby restricting the potential clinical benefit to a limited number of patients, and/or have the ribosome as target.

Identification of new readthrough drugs is thus still of paramount importance, as it will help broadening the medical applications of this therapeutic strategy.

Consequently, there is a need for new PTC readthrough promoters, which would be efficient on a different set of PTC, or with a different spectrum of action.

The present invention fulfils these needs.

Indeed, surprisingly, the inventors have identified a compound, called translectine 68 (TLN68), which does not share any similarity with already known readthrough promoters, and have tested it using the 40 most frequently occurring nonsense mutations responsible for Duchenne myopathy in the DMD gene. As shown in example 1, they identified that it stimulates PTC readthrough over a broad range of nonsense sequences.

Thus, the present invention concerns the use of at least one compound of formula (I), or one of its pharmaceutically acceptable salts, for preventing and/or treating a disease caused by a nonsense mutation, such as a genetic disease or a cancer:

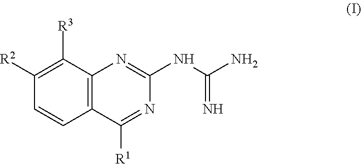

wherein:
R1 is a C1-C6 alkyl radical;
R2 is H, a halogen atom or a C1-C6 alkoxy radical; and
R3 is H, a C1-C6 alkyl radical or a C1-C6 alkoxy radical.

According to the invention, said genetic disease or cancer is caused by a nonsense mutation.

Another object of the invention relates to a compound of formula (II) or one of its pharmaceutically acceptable salts:

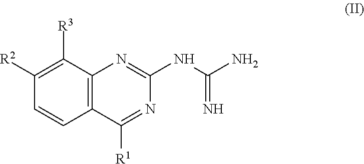

wherein:
R1 is a C1-C6 alkyl radical, preferably methyl or ethyl;
R2 is a halogen atom or a C1-C6 alkoxy radical, preferably methoxy; and
R3 is H or a C1-C6 alkyl radical, preferably methyl,
with the proviso that when R2 is a C1-C6 alkoxy radical, then R1 is ethyl, and
when R2 is a halogen and R3 is H then the compound is in the form of a pharmaceutically acceptable salt such as a salt with formic acid or with iodine.

Another object of the invention is the use of at least one compound of formula (II) or one of its pharmaceutically acceptable salts, in therapy or as a drug (or medicament).

Another object of the invention is a composition comprising, in a pharmaceutically acceptable carrier, at least one compound of formula (II) or one of its pharmaceutically acceptable salts.

Another object of the invention is a product comprising:
a) a compound of formula (I), or one of its pharmaceutically acceptable salts, and
b) at least one other drug,
   as combination product for a simultaneous, separate or sequential use for treating a disease caused by a nonsense mutation, especially a cancer or a genetic disease, in a subject.

Another object of the invention is a product comprising:
a) a compound of formula (I), or one of its pharmaceutically acceptable salts, and
b) at least one chemotherapeutic drug,
   as combination product for a simultaneous, separate or sequential use for treating cancer, and/or for preventing cancer metastasis, and/or for preventing cancer recurrence, and/or for decreasing resistance to the chemotherapeutic drug b), in a subject.

Preferably, the invention relates to a product comprising:
a) a compound of formula (I), or one of its pharmaceutically acceptable salts, and
b) at least one drug chosen from ataluren, gentamicin, negamycin, clitocine, escin and NMD inhibitors,
   as combination product for a simultaneous, separate or sequential use for treating a disease caused by a nonsense mutation, especially a cancer or a genetic disease, in a subject.

Therapeutic Use of Compounds of Formula (I) or their Salts

First, the present invention concerns the use of at least one compound of formula (I), or one of its pharmaceutically acceptable salts, for preventing and/or treating a disease caused by a nonsense mutation, such as a genetic disease or a cancer:

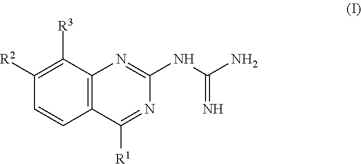

wherein:
R1 is a C1-C6 alkyl radical;
R2 is H, a halogen atom or a C1-C6 alkoxy radical; and
R3 is H, a C1-C6 alkyl radical or a C1-C6 alkoxy radical.

Preferably, the disease is chosen from genetic diseases caused by a nonsense mutation and cancers caused by a nonsense mutation which is present in a tumor-suppressor gene.

By “disease caused by a nonsense mutation”, it is meant that a nonsense mutation responsible for the disease is present in a coding sequence, leading to the production of the corresponding protein in truncated form, and/or to the degradation of the corresponding mRNA by the NMD pathway. The nonsense mutation may be genetically inherited, such as in the case of a genetic disease, or spontaneous, such as it may occur in a cancer.

According to the invention, said genetic disease or cancer is caused by the presence of a nonsense mutation in a coding sequence of interest.

In the case of a genetic disease, the nonsense mutation may be present in genes, such as any one of the genes CFTR (cystic fibrosis transmembrane conductance regulator), DMD (dystrophin), collagen genes (such as COL6A1, COL6A2, COL6A3, COL6A4, COL6A5 and/or COL6A6), SMN1 (survival of motor neuron 1), IDUA (alpha-L-iduronidase), USH1C (USH1 protein network component harmonin). For example, nonsense mutations in SMN1 gene may cause spinal muscular atrophy or amyotrophic lateral sclerosis; nonsense mutations in IDUA may cause a mucopolysaccharidosis type I (MPS I), such as Hurler syndrome (MPS I-H); and nonsense mutations in USH1C may cause Usher syndrome.

In the case of a cancer, the nonsense mutation may be present in a tumor-suppressor gene, such as for example p53, BRCA1, BRCA2 or APC gene.

Compounds of formula (I) are inhibitors of PTC, which are also called PTC readthrough promoters or PTC readthrough inducers.

By “readthrough”, it is meant the event according to which the premature termination codon (PTC) is passed through during translation of a coding sequence, leading to the translation of the corresponding full-length polypeptide. A PTC is created when a nonsense mutation appears in a coding sequence. As a result, the corresponding protein is produced in truncated form, and the mRNA may be degraded by the NMD pathway. Readthrough leads to the translation of a full-length polypeptide. It may be achieved by the entry of a near-cognate tRNA at the PTC introducing an amino acid during translation instead of interrupting translation.

As shown in the examples, the compounds of formula (I) are able to induce PTC readthrough.

By “preventing” or “prevention” of a disease, it is meant alleviating the occurrence of said disease.

By “treatment” or “treating” a disease, it is meant a curative treatment of said disease. A curative treatment is defined as a treatment that completely treat (cure) or partially treat the disease.

The cancer may be any kind of cancer or neoplasia in which the tumor or cancer cells comprise a nonsense mutation. Especially, the cancer is a cancer or neoplasia in which the tumor or cancer cells comprise a nonsense mutation in a tumor-suppressor gene. The cancer is for example selected from squamous cell carcinoma, hepatocellular carcinoma, gastric cancer, oesophageal carcinoma, osteocarcinoma, a melanoma, a breast cancer, a thyroid cancer, a prostate cancer, a colorectal cancer, an ovarian cancer, a lung cancer, a pancreatic cancer, a glioma, a cervical cancer, an endometrial cancer, a head and neck cancer, a liver cancer, a renal cancer, a skin cancer, a testis cancer, an urothelial cancer or an adrenocortical carcinoma, but also non solid cancers such as lymphoma. The cancer can be a metastatic cancer or not.

The genetic disease may be any disease which is due to the presence of a nonsense mutation in a coding sequence of a gene of interest.

It may be chosen from cystic fibrosis (due to a nonsense mutation such as G542X in the cystic fibrosis transmembrane conductance regulator gene), Duchenne muscular dystrophy (due to a nonsense mutation in dystrophin), beta thalassemia (due to nonsense mutation in β-globin), Niemann-Pick disease type A, B or C (due to nonsense L261X mutation in acid sphingomyelinase), Hurler syndrome, Dravet syndrome, spinal muscular atrophy, myoadenylate deaminase deficiency, antithrombin III deficiency, alpha-1 antitrypsin deficiency, apolipoprotein deficiency (apolipoprotein Al, B, CII or E), adenine phosphoribosyltransferase (APRT) deficiency, haemophilia A (due to nonsense mutation in Factor VIII), haemophilia B (due to nonsense mutation in Factor IX), Von Willebrand disease (due to nonsense mutation in Von Willebrand factor), Fanconi anemia-group C, Marfan syndrome, Gaucher disease, Donohue syndrome, oculocerebrorenal syndrome of Lowe, Xeroderma pigmentosum.

The compound of formula (I) according to the invention is preferably in substantially pure form.

By “pharmaceutically acceptable salts”, it is meant any acid addition salts with a halogen, or with inorganic or organic acids, such as formic acid, hydrochloric acid, methanesulfonic acid, hydrobromic acid, hydroiodic acid, sulfuric acid, nitric acid, phosphoric acid, acetic acid, fumaric acid, succinic acid, citric acid, malic acid, tartaric acid, lactic acid or benzoic acid. Preferably, the salt of the compound of formula (I) is a salt with hydrochloric acid, with formic acid or with iodine; preferably with hydrochloric acid. Preferably, the salt of the compound of formula (I) is a salt with hydrochloric acid (i.e. a hydrochloride).

The compound of formula (I) may also comprise at least one isotope, particularly chosen from ²H, ³H, ¹¹C, ¹⁴C, ¹⁸F, ¹⁵O and ¹³N.

By “halogen”, it is meant a fluorine, a chlorine, a bromine or an iodine atom. Preferably, the halogen is a fluorine or chlorine or a bromine.

By “C1-C6 alkyl”, it is meant a linear hydrocarbon group comprising from 1 to 6 carbon atoms, in particular from 1 to 3 carbon atoms, or a branched or cyclic hydrocarbon group comprising from 3 to 6 carbon atoms. Examples of alkyl groups include methyl, ethyl, n-propyl, isopropyl, n-butyl, isobutyl, tert-butyl, n-pentyl, isopentyl, n-hexyl, and cyclohexyl groups, and preferably methyl or ethyl.

A “C1-C6 alkoxy” is an —O-alkyl group wherein the alkyl moiety is a C1-C6 alkyl as defined above. Preferably the C1-C6 alkoxy group is methoxy.

Preferably, the compounds of formula (I) are such that:

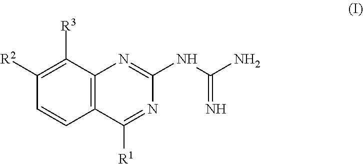

R1 is a C1-C6 alkyl radical, preferably methyl or ethyl;
R2 is H, a halogen atom or a C1-C6 alkoxy radical (preferably methoxy); and
R3 is H, a C1-C6 alkyl radical (preferably methyl) or a C1-C6 alkoxy radical (preferably methoxy),
wherein R2 and R3 are each not simultaneously H.

Preferably, the compounds of formula (I) are such that:

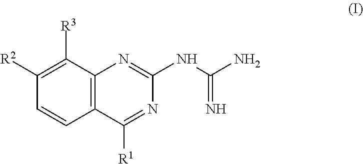

R1 is methyl or ethyl;
R2 is H, a halogen atom or a methoxy radical; and
R3 is H, a methyl radical or a methoxy radical.

Preferably, the compounds of formula (I) are such that:

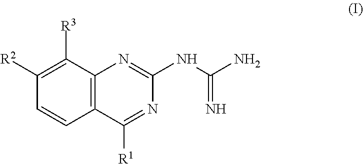

R1 is methyl or ethyl, preferably methyl;
R2 a halogen atom, preferably a fluorine, a chlorine or a bromine atom, or a methoxy radical; and
R3 is H or a methyl radical.

Preferably, the compounds of formula (I) are such that R1 is methyl and R3 is H. According to this embodiment, preferably R2 is H or a C1-C6 alkoxy radical, preferably methoxy.

Preferably, such compounds are chosen from the following compounds and their pharmaceutically acceptable salts:

TLN68, which is

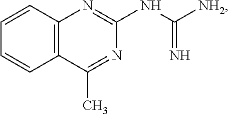

and

EC-18, which is

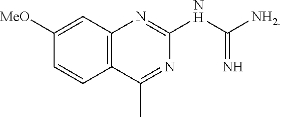 [SMILES: COc1ccc2c(C)nc(NC(=N)N)nc2c1]

Preferably, the compounds of formula (I) are such that R1 is methyl, R3 is H and R2 is a C1-C6 alkoxy radical, preferably methoxy. In such a case, preferably, the compound of formula (I) is EC-18.

Preferably, according to an other embodiment, the compounds of formula (I) are such that R1 is methyl, R2 is H and R3 is a C1-C6 alkyl radical, preferably methyl, or a C1-C6 alkoxy group, preferably methoxy.

Preferably, such compounds are chosen from the following compounds and their pharmaceutically acceptable salts:

EC-35 which is

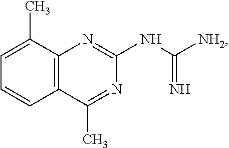 [SMILES: Cc1nc(NC(=N)N)nc2c(C)cccc12]

and

EC-30 which is

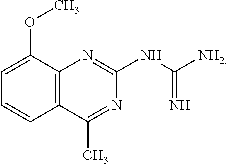 [SMILES: COc1cccc2c(C)nc(NC(=N)N)nc12]

Preferably, according to an other embodiment, the compounds of formula (I) are such that R1 is methyl, R2 is a halogen and R3 is H or a C1-C6 alkyl radical, preferably methyl. Preferably R2 is a fluorine, a chlorine or a bromine atom. Preferably R3 is a C1-C6 alkyl radical, preferably methyl.

Preferably, such compounds are chosen from the following compounds and their pharmaceutically acceptable salts:

EC-141 which is

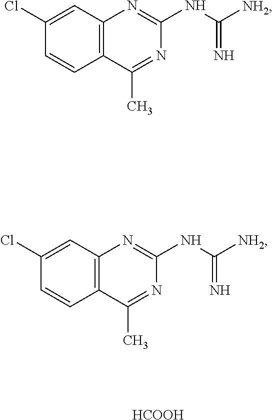 [SMILES: Cc1nc(NC(=N)N)nc2cc(Cl)ccc12 ; Cc1nc(NC(=N)N)nc2cc(Cl)ccc12 ; "HCOOH"]

EC-85 which is

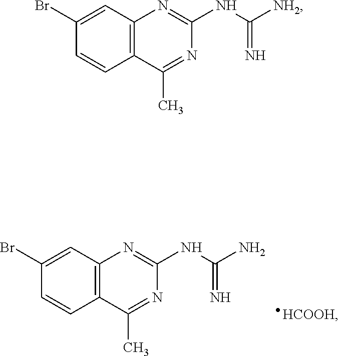 [SMILES: Cc1nc(NC(=N)N)nc2cc(Br)ccc12 ; Cc1nc(NC(=N)N)nc2cc(Br)ccc12 ; "· HCOOH,"]

EC-11 which is

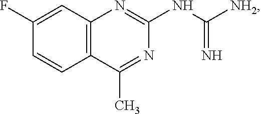 [SMILES: Cc1nc(NC(=N)N)nc2cc(F)ccc12]

EC-288 which is

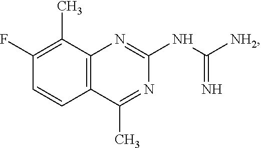 [SMILES: Cc1nc(NC(=N)N)nc2c(C)c(F)ccc12]

EC-130 which is

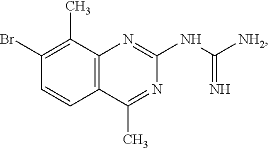 [SMILES: Cc1nc(NC(=N)N)nc2c(C)c(Br)ccc12]

EC-335 which is

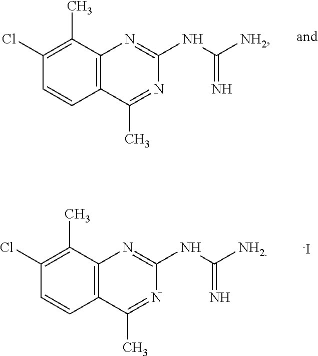 [SMILES: Cc1nc(NC(=N)N)nc2c(C)c(Cl)ccc12 ; Cc1nc(NC(=N)N)nc2c(C)c(Cl)ccc12]

Preferably, according to an other embodiment, the compounds of formula (I) are such that R1 is ethyl, R2 is a C1-C6 alkoxy radical, preferably methoxy, and R3 is H.

Preferably, such compound is chosen from the following compounds and their pharmaceutically acceptable salts:

EC-265 which is

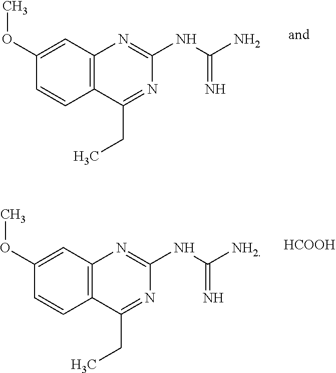 [SMILES: CCc1nc(NC(=N)N)nc2cc(OC)ccc12 ; CCc1nc(NC(=N)N)nc2cc(OC)ccc12 ; "HCOOH"]

Preferably, the compounds of formula (I) or their salts are such that:
R1 is methyl, R3 is H and R2 is a C1-C6 alkoxy radical, preferably methoxy. In such a case, preferably, the compound of formula (I) is EC-18 or one of its salts; or
R1 is methyl, R2 is a halogen, preferably a fluorine, a chlorine or a bromine atom, and R3 is H or a C1-C6 alkyl radical, preferably methyl. In such a case, preferably, the compound of formula (I) is EC-85, EC-288, EC-130, EC-335 or one of their salts; or
R1 is ethyl, R2 is a C1-C6 alkoxy radical, preferably methoxy, and R3 is H. In such a case, preferably, the compound of formula (I) is EC-265 or one of its salts.

More preferably, the compounds of formula (I) or their salts are chosen from:

EC-85 which is

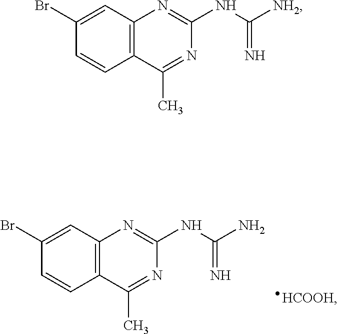 [SMILES: Cc1nc(NC(=N)N)nc2cc(Br)ccc12 ; Cc1nc(NC(=N)N)nc2cc(Br)ccc12 ; "· HCOOH,"]

EC-288 which is

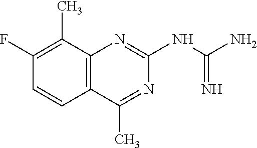 [SMILES: Cc1nc(NC(=N)N)nc2c(C)c(F)ccc12]

and their salts.

Compounds of Formula (II) or their Salts

The invention also relates to a compound of formula (II) or one of its pharmaceutically acceptable salts:

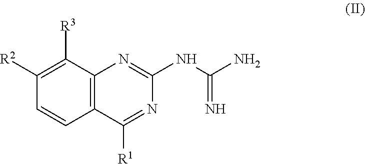

wherein:
R1 is a C1-C6 alkyl radical, preferably methyl or ethyl;
R2 is a halogen atom or a C1-C6 alkoxy radical, preferably methoxy; and
R3 is H or a C1-C6 alkyl radical, preferably methyl,
with the proviso that when R2 is a C1-C6 alkoxy radical, then R1 is ethyl, and
when R2 is a halogen and R3 is H then the compound is in the form of a pharmaceutically acceptable salt such as a salt with formic acid or with iodine.

The compounds of formula (II) are a subgroup of the compounds of formula (I).

All the definitions for the pharmaceutically acceptable salts, the isotope, the halogen, the C1-C6 alkyl radical and the C1-C6 alkoxy radical, are also applicable here for formula (II). Likewise, all the uses of a compound of formula (I) or one of its pharmaceutically acceptable salts are also applicable to the compounds of formula (II).

Preferably, the compounds of formula (II) are such that R1 is methyl, R2 is a halogen and R3 is H or a C1-C6 alkyl radical, preferably methyl.

Preferably, such compounds are chosen from the following compounds and their pharmaceutically acceptable salts:

EC-141 which is

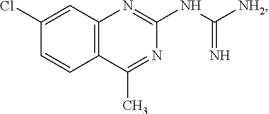 [SMILES: Cc1nc(NC(=N)N)nc2cc(Cl)ccc12]

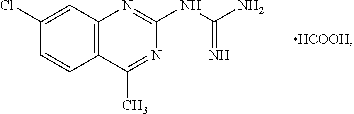 [SMILES: Cc1nc(NC(=N)N)nc2cc(Cl)ccc12]

EC-85 which is

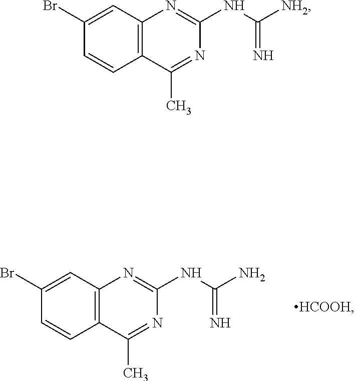 [SMILES: Cc1nc(NC(=N)N)nc2cc(Br)ccc12 ; Cc1nc(NC(=N)N)nc2cc(Br)ccc12]

EC-11 which is

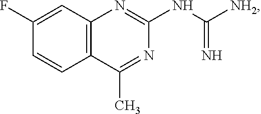 [SMILES: Cc1nc(NC(=N)N)nc2cc(F)ccc12]

EC-288 which is

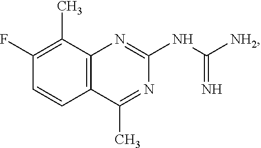 [SMILES: Cc1nc(NC(=N)N)nc2c(C)c(F)ccc12]

EC-130 which is

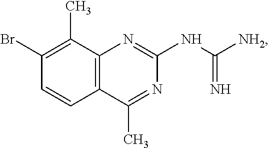 [SMILES: Cc1nc(NC(=N)N)nc2c(C)c(Br)ccc12]

EC-335 which is

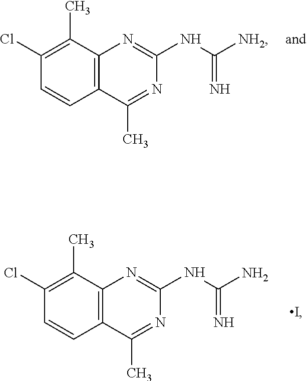 [SMILES: Cc1nc(NC(=N)N)nc2c(C)c(Cl)ccc12 ; "and" ; Cc1nc(NC(=N)N)nc2c(C)c(Cl)ccc12]

Preferably, such compounds are chosen from the following compounds and their pharmaceutically acceptable salts:

EC-141 which is

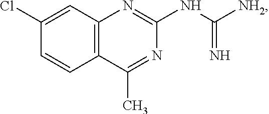 [SMILES: Cc1nc(NC(=N)N)nc2cc(Cl)ccc12]

EC-85 which is

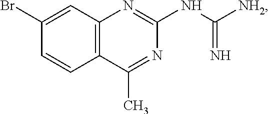 [SMILES: Cc1nc(NC(=N)N)nc2cc(Br)ccc12]

EC-288 which is

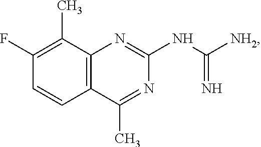 [SMILES: Cc1nc(NC(=N)N)nc2c(C)c(F)ccc12]

EC-130 which is

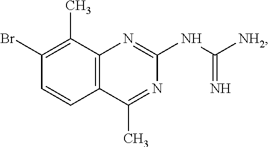 [SMILES: Cc1nc(NC(=N)N)nc2c(C)c(Br)ccc12]

EC-335 which is

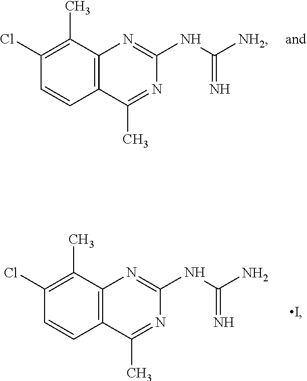 [SMILES: Cc1nc(NC(=N)N)nc2c(C)c(Cl)ccc12 ; "and" ; Cc1nc(NC(=N)N)nc2c(C)c(Cl)ccc12]

Preferably, according to an other embodiment, the compounds of formula (II) are such that R1 is ethyl, R2 is a C1-C6 alkoxy radical, preferably methoxy, and R3 is H.

Preferably, such compound is chosen from the following compounds and their pharmaceutically acceptable salts:

EC-265 which is

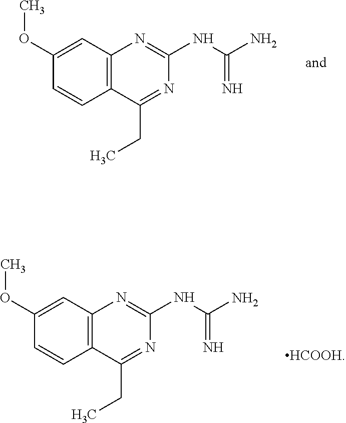 [SMILES: CCc1nc(NC(=N)N)nc2cc(OC)ccc12 ; CCc1nc(NC(=N)N)nc2cc(OC)ccc12]

Another object of the invention is the use of at least one compound of formula (II) or one of its pharmaceutically acceptable salts, in therapy or as a drug (or medicament).

Another object of the invention is a composition comprising, in a pharmaceutically acceptable medium, at least one compound of formula (II) or one of its pharmaceutically acceptable salts.

Preparation of the Compounds of the Invention

The compounds of formula (I) or (II) according to the present invention may be prepared as described in General method A and/or B and as delineated in the following schemes:

General method A. Synthetic route to compounds substituted at C7 and/or C8 positions (Scheme 1):

2,2,4-trimethyl-1,2-dihydroquinolines (1). An oven-dried Schlenk tube equipped with a nitrogen inlet and a magnetic stir bar was sequentially charged with the corresponding aniline (10 mmol), indium (III) chloride (0.5 mmol, 5 mol %) and acetone (12 mL). The mixture was stirred at 56° C. until a full conversion of the starting material was observed, then all volatiles were removed in vacuo and the residue was purified by normal-phase column chromatography using a gradient of EtOAc in cyclohexane.

Translectin 68 derivatives (TLN68) (2). To a solution of the corresponding 2,2,4-trimethyl-1,2-dihydroquinoline 1 (1 mmol) in dry acetonitrile (1 mL) was added 1M HCl in Et₂O (1 equiv.). Upon complete precipitation of the hydrochloride salt of 1, the solids were collected by vacuum suction filtration on glass frit filter and repeatedly washed with hexanes. This material was used in the next step without further purification.

A round-bottom flask equipped with a magnetic stir bar and a reflux condenser was charged with the corresponding hydrochloride salt of 1 (1 mmol), dicyandiamide (1.01 equiv.), EtOH (1 mL) and water (1.5 mL). The mixture was refluxed for 18 h, then cooled to r.t. and the pH was adjusted to 9-10. The precipitate was collected by vacuum suction filtration on glass frit filter and thoroughly washed with cold water. Purification by reverse-phase column chromatography using a gradient of acetonitrile in water supplemented with 0.1% v/v formic acid afforded quinazolyl-2-guanidines 2 as formate salts.

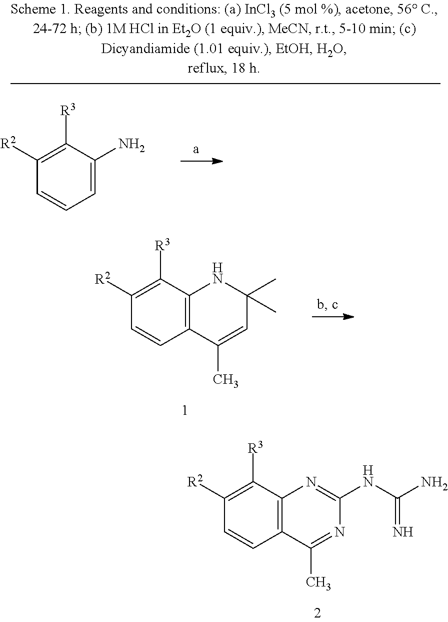

General method B. Synthetic route to compounds substituted at C4 and/or C7-C8 positions (Scheme 2):

ortho-Aminophenones (1). An oven-dried two-neck round bottom flask equipped with a nitrogen inlet and a magnetic stir bar was charged with a solution of the corresponding 2-aminobenzonitrile (5 mmol) in dry THF (15 mL). The corresponding Grignard reagent (3 equiv.) was added dropwise at 0° C. via a syringe. The mixture was stirred for 1 h at 0° C., then allowed to warm to r.t. and stirred at this temperature for 18 h. An aqueous saturated solution of ammonium chloride (15 mL) was added and the biphasic mixture was vigorously stirred for 10 min. The layers were separated and the aqueous phase was extracted twice with EtOAc. The combined organics were dried over sodium sulfate, concentrated in vacuo and the residue was purified by normal-phase column chromatography using a gradient of EtOAc in cyclohexane.

Translectin 68 derivatives (2). A round-bottom flask equipped with a magnetic stir bar and a reflux condenser was charged with the corresponding ortho-aminophenone 1 (1 mmol), dicyandiamide (1.05 equiv.), pTSA (5 mol %) and DMF (3.5 mL). The mixture was stirred at 120° C. until a full conversion of the starting material was observed, then cooled to r.t., diluted with EtOAc and repeatedly washed with water and brine. All volatiles were removed in vacuo and the residue was purified by reverse-phase column chromatography using a gradient of acetonitrile in water supplemented with 0.1% v/v formic acid to afford quinazolyl-2-guanidines 2 as formate salts.

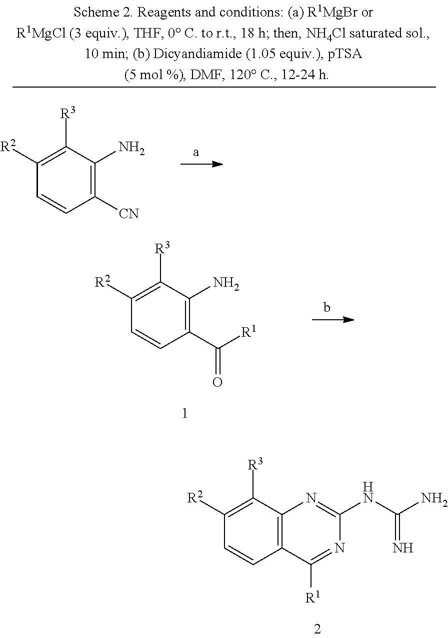

Therapeutic Combinations with the Compounds of the Invention

Another object of the invention is a product comprising:
a) a compound of formula (I) or (II), or one of its pharmaceutically acceptable salts, and
b) at least one other drug,
   as combination product for a simultaneous, separate or sequential use for treating a disease caused by a nonsense mutation, especially a cancer or a genetic disease, in a subject. Preferably the disease is chosen from genetic diseases caused by a nonsense mutation and cancers caused by a nonsense mutation which is present in a tumor-suppressor gene.

Another object of the invention is a product comprising:
a) a compound of formula (I) or (II), or one of its pharmaceutically acceptable salts, and
b) at least one chemotherapeutic drug,
   as combination product for a simultaneous, separate or sequential use for treating cancer, and/or for preventing cancer metastasis, and/or for preventing cancer recurrence, and/or for decreasing resistance to the chemotherapeutic drug b), in a subject.

Preferably, the invention relates to a product comprising:
a) a compound of formula (I) or (II), or one of its pharmaceutically acceptable salts, and
b) at least one drug chosen from ataluren, gentamicin, negamycin, clitocine, escin and NMD inhibitors,
   as combination product for a simultaneous, separate or sequential use for treating a disease caused by a nonsense mutation, especially a cancer or a genetic disease, in a subject. Preferably the disease is chosen from genetic diseases caused by a nonsense mutation and cancers caused by a nonsense mutation which is present in a tumor-suppressor gene.

The term “subject” refers to any subject and typically designates a patient, in particular a subject undergoing a treatment of a genetic disease, or of a cancer such as chemotherapy and/or radiotherapy, or a subject at risk, or suspected to be at risk, of developing a cancer.

The subject is preferably a mammal, even more preferably a human being. For example, the subject may be a human being suffering of a cancer.

The subject may have been exposed to part of a complete conventional treatment protocol, for example to at least one cycle of the all treatment protocol, for example two cycles of the all treatment protocol.

Typically, the other drug may be a drug used for treating a genetic disease or a cancer. In a particular embodiment of the present invention, the other drug is another PTC readthrough inducer, preferably chosen from ataluren, gentamicin, negamycin, clitocine and escin. In another particular embodiment of the present invention, the other drug is chosen from NMD inhibitors.

Indeed, the compounds of formula (I) or (II) of the invention may not target the ribosome. Thus, they could be used in combination with previous readthrough inducers, such as ataluren, gentamicin, negamycin, clitocine or escin, which target the ribosome.

The compounds of formula (I) or (II) of the invention could also be used in combination with NMD inhibitors. NMD (nonsense-mediated mRNA decay) inhibitors are compounds that decrease the activity of NMD in a cell and/or decrease the destruction of defective mRNA by any measurable amount, as compared to such cell in absence of inhibition. NMD inhibition can be achieved in various ways, e.g. by blocking function of protein components of NMD pathway, by inhibiting translation, or by allowing the translation machine to by-pass the premature termination codon (readthrough).

The NMD inhibitor may be chosen from wortmannin, caffeine, pateamine A, NMDI-1 and 5-azacytidine.

In a particular embodiment of the present invention, the other drug is a chemotherapeutic drug.

In a particular embodiment of the present invention, the chemotherapeutic drug is selected from an anthracycline, an antitumor antibiotic, an alkylating agent, an antimetabolite, an alkaloid, a topoisomerase inhibitor, an anti-mitotic agent such as a spindle poison, a DNA-intercalating agent, a taxane, a platin-based component, a specific kinase inhibitor, an androgen receptor antagonist, an hormone, a cytokine, an antiangiogenic agent, an antibody, in particular a monoclonal antibody, a modulator of the immunity system, an oncolytic virus and a TLR (Toll-Like Receptor)-3 ligand.

The chemotherapeutic drug may be selected depending on the specific cancer to be prevented or treated.

Anthracyclins include for example doxorubicin, daunorubicin, epirubicin, pirarubicin, idarubicin, zorubicin, aclarubicin, nemorubicin, sabarubicin or valrubicin.

Antitumor antibiotics include for example Bleomycin, hydroxyurea, Mitomycin C or Mitoxantrone.

Alkylating agents include for example dacarbazine, busulfan, carboplatin, chlorambucil, cisplatin, cyclophosphamide, ifosfamide, melphalan, mechlorethamine, oxaliplatin, uramustine or temozolomide.

Examples of antimetabolites are Azathioprine, Capecitabine, Cytarabine, Floxuridine, Fludarabine, Fluorouracil, Gemcitabine, Methotrexate, Fluorouracil (5-FU) or Pemetrexed; Alkaloids include for example vinblastine, or vincristine (Vinorelbine);

Topoisomerase inhibitors include, for example Irinotecan, Topotecan or Etoposide; Spindle poisons are for example selected from Vinblastine, Vincristine and Vinorelbine; Taxanes are for example selected from docetaxel, larotaxel, cabazitaxel, paclitaxel (PG-paclitaxel and DHA-paclitaxel), ortataxel, tesetaxel, and taxoprexin.

Examples of platin-based components are CDDP and OXP.

Examples of specific kinase inhibitors are for example BRAF kinase inhibitors such as vemurafenib and dabrafenib, or MEK inhibitors such as trametinib, or Plk1 inhibitors such as volasertib.

Androgen receptor antagonists are for example bicalutamide or enzalutamide.

Tamoxifen and anti-aromatase drugs are typically used in the context of hormonotherapy.

Examples of cytokines usable in the context of an immunotherapy are IL-2 (Interleukine-2) and IFN (Interferon) alpha (IFNa).

Antiangiogenic agents are for example VEGF inhibitors such as itraconazole, bevacizumab or ranibizumab.

Anti-CD20 (pan B-Cell antigen) and anti-Her2/Neu (Human Epidermal Growth Factor Receptor-2/NEU) are examples of monoclonal antibodies. Monoclonal antibodies also include anti-immune checkpoint antibodies, such as anti-PD1, anti-PDL1, anti-CTLA4, anti-OX40L, anti-PDL2, anti-CD73, anti-CD80, anti-CD86, anti-TIGIT, anti-Galactin-3 or anti-HVEM antibodies.

Anti-PD1 antibodies include pembrolizumab or nivolumab.

Immunity system modulators are for example IDO1, IDO2 or TDO2 inhibitors, A2a antagonists or STING agonists.

Oncolytic viruses are for exemple Talimogene laherparepvec.

In a preferred embodiment, the chemotherapeutic drug is selected from cisplatin, doxorubicin, docetaxel, cyclophosphamide, oxaliplatin, irinotecan, methotrexate, temozolomide, 5-FU, dacarbazine and vemurafenib.

Herein described are also a method for treating a disease caused by a nonsense mutation, comprising administering to a subject in need thereof with an effective amount of at least one compound of formula (I) or (II) as defined above, optionally together with an other drug.

As used herein, “a therapeutically effective amount or dose” refers to an amount of the compound of the invention which removes, slows down the cancer or reduces or delays one or several symptoms or disorders caused by or associated with said disease in the subject, preferably a human being. The effective amount, and more generally the dosage regimen, of the compound of the invention and pharmaceutical compositions thereof may be determined and adapted by the one skilled in the art. An effective dose can be determined by the use of conventional techniques and by observing results obtained under analogous circumstances. The therapeutically effective dose of the compound of the invention will vary depending on the disease to be treated or prevented, its gravity, the route of administration, any co-therapy involved, the patient's age, weight, general medical condition, medical history, etc.

Typically, the amount of the compound to be administrated to a patient may range from about 0.01 to 500 mg/kg of body weight for a human patient. In a particular embodiment, the pharmaceutical composition according to the invention comprises 0.01 mg/kg to 300 mg/kg of the compound of the invention, preferably from 0.01 mg/kg to 3 mg/kg, for instance from 25 to 300 mg/kg.

In a particular aspect, the compounds of the invention can be administered to the subject by parenteral route, topical route, oral route or intravenous (IV) injection. The compound or the nanoparticle of the invention may be administered to the subject daily (for example 1, 2, 3, 4, 5, 6 or 7 times a day) during several consecutive days, for example during 2 to 10 consecutive days, preferably from 3 to 6 consecutive days. Said treatment may be repeated during 1, 2, 3, 4, 5, 6 or 7 weeks, or every two or three weeks or every one, two or three months. Alternatively, several treatment cycles can be performed, optionally with a break period between two treatment cycles, for instance of 1, 2, 3, 4 or 5 weeks. The compound or the nanoparticle of the invention can for example be administered as a single dose once a week, once every two weeks, or once a month. The treatment may be repeated one or several times per year.

Doses are administered at appropriate intervals which can be determined by the skilled person. The amount chosen will depend on multiple factors, including the route of administration, duration of administration, time of administration, the elimination rate of the selected compound of formula (I), or of the various products used in combination with said compound, the age, weight and physical condition of the patient and his/her medical history, and any other information known in medicine.

The administration route can be oral, topical or parenteral, typically rectal, sublingual, intranasal, intra-peritoneal (IP), intra-venous (IV), intra-arterial (IA), intra-muscular (IM), intra-cerebellar, intrathecal, intratumoral and/or intradermal. The pharmaceutical composition is adapted for one or several of the above-mentioned routes. The pharmaceutical composition is preferably administered by injection or by intravenous infusion of suitable sterile solutions, or in the form of liquid or solid doses via the alimentary canal.

The formulations of the present invention comprise a compound of formula (I) or (II) in a pharmaceutically acceptable carrier. The carrier must be “acceptable” in the sense of being compatible with the other ingredients of the formulations and not deleterious to the recipient thereof.

The pharmaceutical composition can be formulated as solutions in pharmaceutically compatible solvents or as gels, oils, emulsions, suspensions, or dispersions in suitable pharmaceutical solvents or vehicles, or as pills, tablets, capsules, powders, suppositories, etc. that contain solid vehicles in a way known in the art, possibly through dosage forms or devices providing sustained and/or delayed release. For this type of formulation, an agent such as cellulose, lipids, carbonates or starches are used advantageously.

Agents or vehicles that can be used in the formulations (liquid and/or injectable and/or solid) are excipients or inert vehicles, i.e. pharmaceutically inactive and non-toxic vehicles. Mention may be made, for example, of saline, physiological, isotonic and/or buffered solutions, compatible with pharmaceutical use and known to those skilled in the art. The compositions may contain one or more agents or vehicles chosen from dispersants, solubilizers, stabilizers, preservatives, etc.

Particular examples are methylcellulose, hydroxymethylcellulose, carboxymethylcellulose, cyclodextrins, polysorbate 80, mannitol, gelatin, lactose, liposomes, vegetable oils or animal, acacia, etc. Preferably, vegetable oils are used.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations suitable for parenteral administration conveniently comprise a sterile oily or aqueous preparation of the active ingredient which is preferably isotonic with the blood of the recipient. Every such formulation can also contain other pharmaceutically compatible and non-toxic auxiliary agents, such as, e.g. stabilizers, antioxidants, binders, dyes, emulsifiers or flavouring substances.

Further aspects and advantages of the present invention will be disclosed in the following experimental section which shall be considered as illustrative only.

### LEGENDS TO THE FIGURES

Note that in the figures, TLN68 is the same molecule as “TLN468”.

FIG. 1: Primary selection and Secondary quantification of the readthrough effect of the positive hits

A) Positives hits were selected according to their strictly standardized mean difference (SSMD) score and according to the increase factor of the luciferase activity between the treated condition and the median of the negative controls (untreated cells). The molecules from the Chembridge chemotherapy library and those of Prestwick are represented in left and right panels respectively. The red crosses correspond to the molecules whose SSMD ≥2 or the factor of increase is ≥1.4 for a total of 465 molecules.

B) The dual reporter system is indicated in the top part. The stop indicates the R213X sequence inserted between lacZ and F-luc. The right part shows the ability of the molecules to stimulate readthrough. NIH3T3 cells were treated for 24 hours with the gentamicin (2.5 mM) as control or with the 43 molecules selected (50 μM). Arrows represent the 4 molecules (TLN1399, TLN236, TLN309 and TLN68) inducing readthrough by a factor greater than 4.

FIG. 2: Western blots from HDQ-P1 cells harboring the endogenous nonsense mutation P53-R213X

A) The p53 protein is detected by the p53-DO1 antibody recognizing the N-terminal region. Actin is used as control. Control was only obtained in one of two Western blots.

B) HDQ-P1 cells were treated or not with G418 (400 μM), TLN68 (80 μM) or TLN309 (40 μM) for 48 hours. Mutant p53 mRNA levels were determined by quantitative PCR (n=3).

The results of each condition are expressed in relation to the normalized relative amount of mRNA in the absence of a treatment.

C) Structures of the TLN68 (Translectine, or TLN468) and TLN309 (Amodiaquine).

FIG. 3: TLN68 restores p53-R231X activity

A) The human H1299 (P53^{-/-}) cells were cotransfected by the vectors pCMV-R213X, pCMV-LacZ and p53BS-luc. The pCMV-R213X vector expresses the P53 gene with the nonsense R213X mutation. The p53BS-luc vector expresses the luciferase gene under the control of a p53-dependent promoter and allow detection of a transcriptionally active p53. The vector pCMVLacZ was used to normalize the results.

B) The HDQ-P1 cells were treated or not with G418 (400 μM) or TLN68 (80 μM) for 48 hours. Bax gene mRNA levels, one of the major targets of p53, were determined by quantitative PCR (n=3). The results of each condition are expressed relative to the normalized relative amount of mRNA in the absence of a treatment set at 1.

FIG. 4: Sequence specificity of TLN68

A) Schematic representation of the dystrophin gene and the position of the selected 40 mutations.

B) Quantification of stop codon readthrough efficiency for the 40 more frequent nonsense mutations found in DMD gene (n=6), in presence or absence of TLN68 at 60 mM.

Gentamicin (2.5 mM) measurement has been done in the same conditions to compare the TLN68 effect with gentamicin.

FIG. 5: Additive effect between TLN68 and gentamicin

Hela cells are treated with gentamicine, TLN68 or both drugs during 24 h before stop codon readthrough quantification. The values for the untreated cells are setup to 1 and fold changes are calculated from this value. At least six independent measurements are performed for each condition.

FIG. 6: Selection of the recombinant cell-line for HTS

Recombinant NIH3T3 cells are cultured from 96-wells plates starting from 15,000 cells/well. Luciferase activity is quantified by adding directly the coelenterazine in the well (n=4). Black and white bars represent basal metridia-luciferase activity and gentamicine (1.6 mM) induced activity, respectively. The inventors retain the clone number C14 for further development.

FIG. 7: Statistical validation of the reporter cell line SSMD values obtained for the screening of the two libraries Chembridge and Prestwick are in panels A and B respectively. X-crosses indicate compound with a SSMD>2, † crosses represent negative controls added to the plates during the screening.

FIG. 8: TLN68 has no impact on translational frameshifting

TLN468 has no impact on both −1 and +1 programmed ribosomal frameshifting (PRF). No significant difference is observed between treated and untreated cells (right and left bars respectively). TLN468 has been tested on −1 PRF from HIV-1 and +1 PRF from OAZ1 gene.

FIG. 9: WST1 assay to quantify toxicity of TLN468 on the human cell line HELA, and the mouse cell line NIH3T3.

### EXAMPLE 1: PREPARATION OF COMPOUNDS OF FORMULA (I) OR (II) ACCORDING TO THE INVENTION

The following examples are prepared according to the protocols described above:

### 1-(7-methoxy-4-methylquinazolin-2-yl)guanidine EC-18 (C₁₁H₁₃N₅O, 231.26 g/mol)

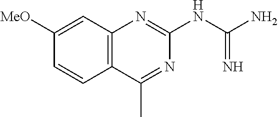 [SMILES: COc1ccc2c(C)nc(NC(=N)N)nc2c1]

¹H NMR (400 MHz, (CD₃)₂SO): 8.45 (s, 1H), 8.08 (d, J=9.1 Hz, 1H), 7.35 (d, J=2.5 Hz, 1H), 7.15 (dd, J=9.1, 2.6 Hz, 1H), 3.93 (s, 3H), 2.79 (s, 3H).

¹³C NMR (400 MHz, (CD₃)₂SO): 169.7, 167.4, 164.2, 156.4, 155.5, 151.4, 127.5, 117.7, 115.8, 105.5, 55.8, 21.3.

IR (neat): 3240 (broad), 2974, 1686, 1562, 1340, 1215, 1018, 699.

HRMS calcd for C₁₁H₁₄N₅O (M+H⁺): 232.1193 Found: 232.1194.

### 1-(4,8-dimethylquinazolin-2-yl)guanidine EC-35 (C₁₁H₁₃N₅, 215.26 g/mol)

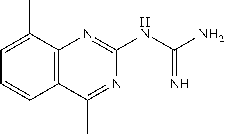 [SMILES: Cc1nc(NC(=N)N)nc2c(C)cccc12]

¹H NMR (400 MHz, (CD₃)₂SO): 9.23 (br s, 3H), 8.45 (s, 1H), 8.03 (d, J=8.2 Hz, 1H), 7.79 (d, J=7.0 Hz, 1H), 7.49-7.42 (m, 1H), 2.86 (s, 3H), 2.55 (s, 3H).

¹³C NMR (400 MHz, (CD₃)₂SO): 171.6, 167.6, 156.6, 154.2, 147.5, 134.6, 133.7, 125.1, 123.7, 120.4, 21.7, 17.2.

IR (neat): 3210 (broad), 2981, 1697, 1586, 1353, 1153, 759.

HRMS calcd for C₁₁H₁₄N₅ (M+H⁺): 216.1244 Found: 216.1246.

### 1-(7-bromo-4,8-dimethylquinazolin-2-yl)guanidine EC-130 (C₁₁H₁₂BrN₅, 294.16 g/mol)

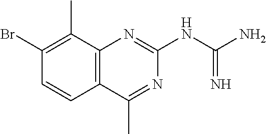 [SMILES: Cc1nc(NC(=N)N)nc2c(C)c(Br)ccc12]

¹H NMR (400 MHz, (CD₃)₂SO): 8.42 (s, 1H), 7.93 (d, J=8.8 Hz, 1H), 7.67 (d, J=8.9, 1H), 2.83 (s, 3H), 2.63 (s, 3H).

¹³C NMR (400 MHz, (CD₃)₂SO): 171.4, 166.9, 157.2, 156.3, 148.7, 133.3, 130.0, 128.4, 124.9, 119.4, 21.7, 17.1.

IR (neat): 3305 (broad), 2972, 1694, 1560, 1323, 1032, 780.

HRMS calcd for C₁₁H₁₃BrN₅ (M+H⁺): 294.0349 Found: 294.0350.

### 1-(7-fluoro-4,8-dimethylquinazolin-2-yl)guanidine EC-288 (C₁₁H₁₂FN₅, 233.25 g/mol)

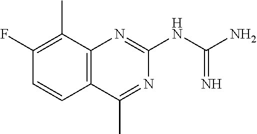 [SMILES: Cc1nc(NC(=N)N)nc2c(C)c(F)ccc12]

¹H NMR (400 MHz, (CD₃)₂SO): 8.41 (s, 1H), 8.11 (d, J=9.1, 6.1 Hz, 1H), 7.41 (d, J=9.3, 1H), 2.83 (s, 3H), 2.41 (s, 3H).

¹³C NMR (400 MHz, (CD₃)₂SO): 171.5, 167.2, 164.4, 161.9, 156.0, 155.1, 149.5, 126.0 (³J_{CF}=10.8 Hz), 117.9 (²J_{CF}=16.1 Hz), 115.0 (²J_{CF}=26.5 Hz), 38.9 (overlapping with the solvent residual peak), 8.58 (³J_{CF}=4.0 Hz).

¹⁹F (376 MHz, (CD₃)₂SO): −106.5.

IR (neat): 3318 (broad), 2922, 1695, 1587, 1336, 1059, 788.

HRMS calcd for C₁₁H₁₃FN₅ (M+H⁺): 234.1150 Found: 234.1153.

### 1-(7-chloro-4,8-dimethylquinazolin-2-yl)guanidine EC-335 (C₁₁H₁₂ClN₅, 249.70 g/mol)

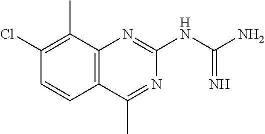 [SMILES: Cc1nc(NC(=N)N)nc2c(C)c(Cl)ccc12]

¹H NMR (400 MHz, (CD₃)₂SO): 8.41 (s, 1H), 8.02 (d, J=8.9 Hz, 1H), 7.52 (d, J=8.9 Hz, 1H), 2.84 (s, 3H), 2.60 (s, 3H).

¹³C NMR (400 MHz, (CD₃)₂SO): 170.9, 166.6, 157.4, 149.0, 138.3, 131.0, 125.4, 124.8, 124.7, 118.7, 21.7, 14.0.

IR (neat): 3301 (broad), 2970, 1694, 1571, 1325, 1022, 782.

HRMS calcd for C₁₁H₁₃ClN₅ (M+H⁺): 250.0854 Found: 250.0858.

### Example 2: Translational Suppression of PTC Mutations in Duchenne and Becker Muscular Dystrophy (DMD)

Materials and Methods

Cell Lines and Plasmids

All cells were cultured in DMEM plus GlutaMAX (Invitrogen), except for H1299 cells, which were cultured in RPMI plus GlutaMAX (Invitrogen). The medium was supplemented with 10% foetal calf serum (FCS, Invitrogen) and 100 U/ml penicillin/streptomycin. Cells were kept in a humidified atmosphere containing 5.5% CO₂, at 37° C. NIH3T3 cells are embryonic mouse fibroblasts. H1299 is a p53-null cell line established from a human lung carcinoma (provided by the ATCC). HDQ-P1 is homozygous for a nonsense mutation at codon 213 (CGA to TGA) in the p53 gene. This cell line was established from a human primary breast carcinoma and was provided by DSMZ-German collection of microorganisms and cell cultures. In order to generate a stable mammalian cell line the inventors used a secreted Metridia luciferase reporter gene derived from pMetLuc2 (Clonetech). The coding sequence of this gene was interrupted by a TP53 nonsense mutation R213X with its own nucleotide context and cloned at an Eco53Kl site created by directed mutagenesis at nucleotide 57. The final construction was named pML213 and was used to stably transfect NIH3T3 cells with JetPei reagent (Invitrogen). Seven neomycin resistant clones were tested for their capacity to express active metridiae after readthrough induction in the presence of 1.6 mM gentamicin during 24 hours. Then 50 μL of culture medium was taken from each well and incubated in the presence of substrate: coelenterazine, according to the conditions recommended by the supplier (Ready-To-Glow Secreted Luciferase Reporter Assay (Clonetech)). The photon emission generated by the reaction was measured in a plate luminometer (Tecan).

The clone presenting the best increase factor between the treated condition and control was chosen to realize the HTS.

HTS Screening

Each molecule from the two libraries was tested at 50 μM. In each plate 8 wells over 80 were reserved for positive control (gentamicin) and 8 wells for negative control (DMSO). The Prestwick Chemical Library is a unique collection of 1,280 small molecules already approved by FDA, EMA and other agencies. The second library is a subgroup of 16,480 compounds, selected on the criteria of chemical and pharmacological diversity from the ChemBridge library that includes more than one million so far. The inventors also checked for false positive hits by testing all selected drugs on NIH3T3 parental cell line without reporter gene in order to eliminate drugs capable of artificially increasing the photon emission.

Statistical Validation of the New Reporter Cell Line

To validate the screening strategy the inventors first applied the screening protocol to five 96 wells plates from the library using gentamicin as positive control and DMSO as negative control. For statistical validation the inventors used the SSMD parameter presented by Zhang et al. in 1999. The strictly standardized mean difference (SSMD) is robust to both measurement unit and strength of positive control. It takes into account data variability in both compared groups and has a probability interpretation. The inventors obtain SSMD 2 validating the screening protocol (FIG. 7).

Readthrough Quantification

Complementary oligonucleotides corresponding to TP53 R213X nonsense mutations and 9 nucleotides on both sides are ligated into the pAC99 dual reporter plasmid, as previously described (Premature stop codons involved in muscular dystrophies show a broad spectrum of readthrough efficiencies in response to gentamicin treatment, Bidou et al, Gene Therapy 1 Apr. 2004, 11(7):619-627). This dual reporter is used to quantify stop-codon readthrough through the measurement of luciferase and beta-galactosidase (internal calibration) activities, as previously described (Stahl et al, Versatile vectors to study recoding: conservation of rules between yeast and mammalian cells. Nucleic Acids Res 1995; 23:1557-60). Readthrough levels for nonsense mutations were analysed in the presence or absence of tested molecules. Cells were seeded in a 6-well plate. The next day, cells are transfected with the reporter plasmid in the presence of JetPei reagent (Invitrogen). The following day, they are rinsed with fresh medium, with or without readthrough inducers. Cells were harvested 24 hours later, with trypsin-EDTA (Invitrogen), lysed with Glo lysis buffer (Promega) and beta-galactosidase and luciferase activities were assayed as previously described (Stahl et al, Versatile vectors to study recoding: conservation of rules between yeast and mammalian cells. Nucleic Acids Res 1995; 23:1557-60). Readthrough efficiency was estimated by calculating the ratio of luciferase activity to beta-galactosidase activity obtained with the test construct, with normalisation against an in-frame control construct. At least five independent transfection experiments were performed for each assay.

RNA Extraction and RT-qPCR

For the analysis of mRNA levels for p53 and its transcriptional target genes, Bax, the inventors extracted total RNA from HDQ-P1 cells that had or had not been treated with G418 (400 μM) or TLN68 (80 μM) for 48 hours (RNeasy Mini Kit, Qiagen). The RNA was treated with DNAse I (RNase-free DNase) and quantified with a Nanodrop spectrometer (ThermoScientific). The absence of RNA degradation was confirmed by agarose gel electrophoresis. The first-strand cDNA was synthesized from 2 μg of total RNA, with random primers and the SuperScript II Reverse Transcriptase (Invitrogen), as recommended by the manufacturer. Quantitative PCR was then carried out on equal amounts of the various cDNAs, with a CFX96 thermocycler (Biorad), and the accumulation of products was monitored with the intercalating dye FastStart Universal SYBRGreen Master (ROX) reagent (Roche). The inventors quantified mRNA levels relative to three reference mRNAs: RPL32, Hprt1 and HMBS. In each experiment, results are expressed relative to those for untreated cells, for which the value obtained was set to 1. Relative levels of gene expression were calculated at early stages of PCR, when the amplification was exponential and might, therefore, be correlated with the initial number of copies of the transcript. The specificity of quantitative PCR was checked by agarose gel electrophoresis, which showed that a single product of the desired length was produced for each gene. A melting curve analysis was also performed. Single product-specific melting temperatures were identified for each gene. For the quantification of each mRNA, three independent experiments (from biological replicates) were performed in triplicate. The inventors used the following oligonucleotides pairs for amplification:

p53 forward: 5′CCGCAGT CAGATCCTAGCG 3′ (SEQ ID NO:1) and reverse: 5′CCATTGCTTGGGACGGCAAGG 3′ (SEQ ID NO:2);
Bax forward: 5′GCTGTTG GGCTGGATCCAAG 3′ (SEQ ID NO:3) and reverse 5′ TCAGCCCATCTTCTTCCAGA (SEQ ID NO:4).

Western-Blot Analysis

HDQ-P1 cells (R213X) were treated with G418 (10 and 20 mM), or the selected molecules (TLN68 50 μM, TLN1399 50 μM, TLN236 30 μM and TLN309 40 μM), for 48 h. Cells were harvested by treatment with trypsin—EDTA (Invitrogen), lysed in 350 mM NaCl, 50 mM Tris—HCl pH 7.5, 1% NP-40, and protease inhibitor cocktail (Roche) and disrupted by passage through a syringe.

TLN1399 (out of the present invention) has the following structure:

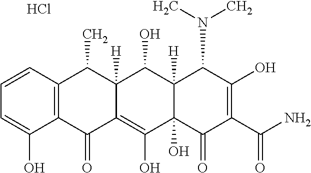 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21 ; Cl]

TLN236 (out of the present invention) has the following structure:

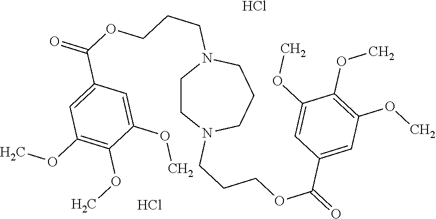

Total proteins were quantified with Bradford reagent (Biorad) and extracts were denatured by incubation in Laemmli buffer for 5 minutes at 90° C. The inventors subjected 30 μg of total protein from HDQ-1 cells to SDS—PAGE in 4/10% Bis—Tris gels. Proteins were transferred onto nitrocellulose membranes, according to the manufacturer's instructions (Biorad). Membranes were saturated by overnight incubation in 5% skimmed milk powder in PBS, and incubated for 1 hour with the primary monoclonal antibody, DO-1 (N-terminal epitope mapping between amino acid residues 11 and 25 of p53; Santa Cruz Biotechnologies, 1/400) or a monoclonal antibody against mouse actin (Millipore, 1/2000). After three washes in PBS supplemented with 0.1% Tween, the membranes were incubated with the secondary antibody [horseradish peroxidase-conjugated anti-mouse IgG ( 1/2500)] for 45 minutes. The membranes were washed five times and chemiluminescence was detected with ECL Prime Western Blotting Detection Reagents (Amersham, GE Healthcare). The signal was quantified with ImageJ software.

TP53 Protein Activity Assays

The inventors investigated the transcriptional activity of the p53 protein in H1299, a p53-null cell line. Cells were cotransfected, by the JetPei method, with the p53BS-luc reporter plasmid containing the firefly luciferase gene downstream from seven p53 binding sites, the pCMVLacZ and the pCMVp53R213X containing the p53 cDNA interrupted by the stop mutation R213X. TLN68 (20, 50, 80 and 100 mM) was added to the medium just before transfection for a total of 20 hours of treatment. Protein extracts were then prepared and enzymatic activities were measured. Transfection with pCMVLacZ was used to normalise transfection efficiency, cell viability and protein extraction. At least six independent transfection experiments were performed for each set of conditions.

Results

Development and Validation of Stable Cell Line for Luciferase Assay

In order to identify new readthrough compounds that are not related to already known drugs the inventors choose an HTS approach. To select the drugs on their readthrough activity, the inventors first generated a stable mammalian cell line (from NIH3T3) by integrating a secreted Metridia luciferase reporter gene interrupted by a nonsense mutation (R213X). This system combines the advantages of a live-cell assay with the sensitivity of an enzyme-based system. The coding sequence of this gene is interrupted by a TP53 nonsense mutation R213X embedded in its own nucleotide context. This mutation has the advantage of presenting an easily measurable basal readthrough level.

The inventors reasoned that if the expression of luciferase is actually due to the readthrough of R213X stop codon, the addition of gentamicin should significantly increases the activity of luciferase. So they tested the 7 independent clones with a stable integration of the reporter gene with 1.6 mM of gentamicine. A significant induction was obtained for all of the clones, and they selected the one displaying the strongest induction (C14) for further development (FIG. 6).

Screening of Two Chemical Libraries

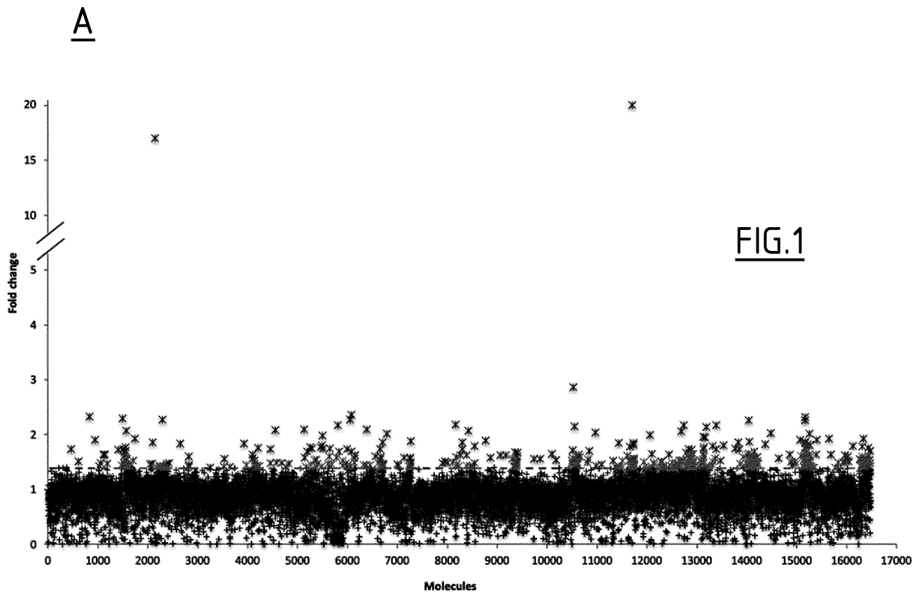

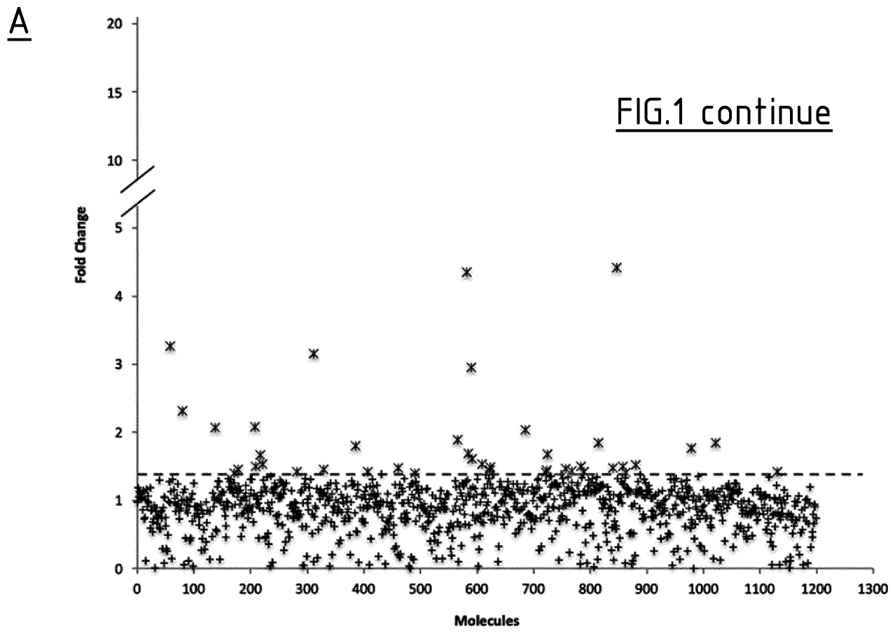

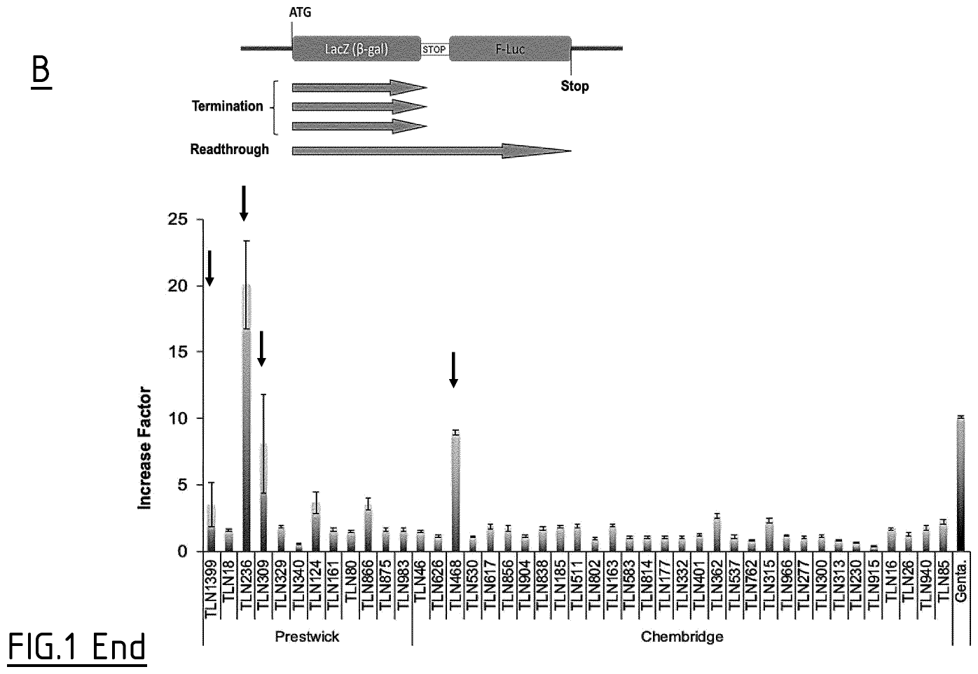

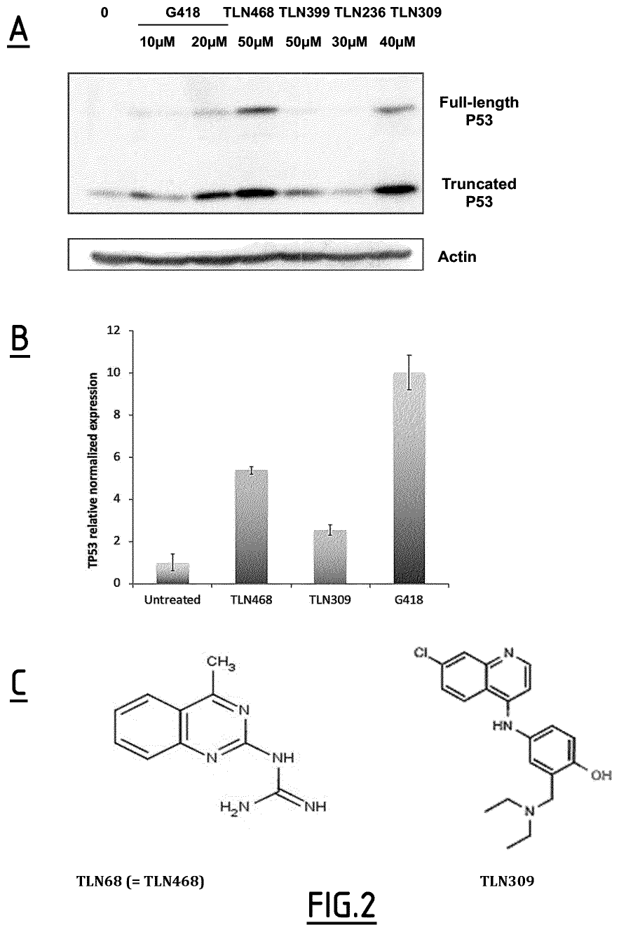

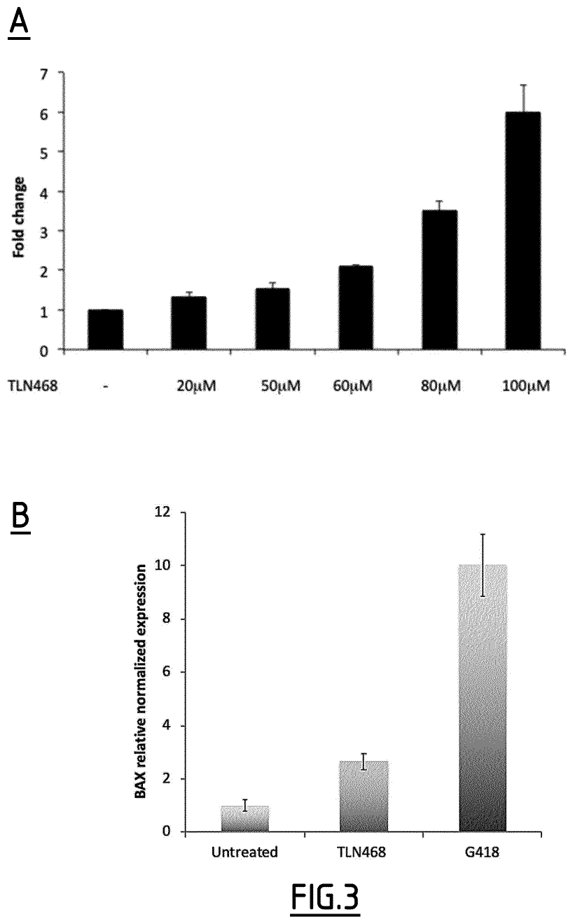

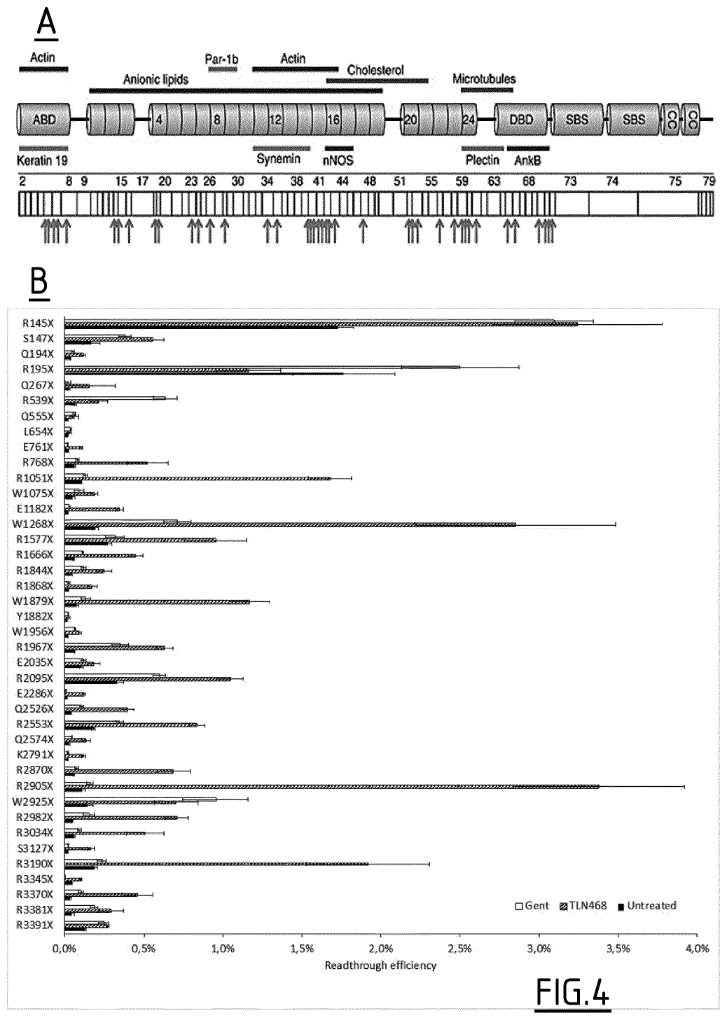

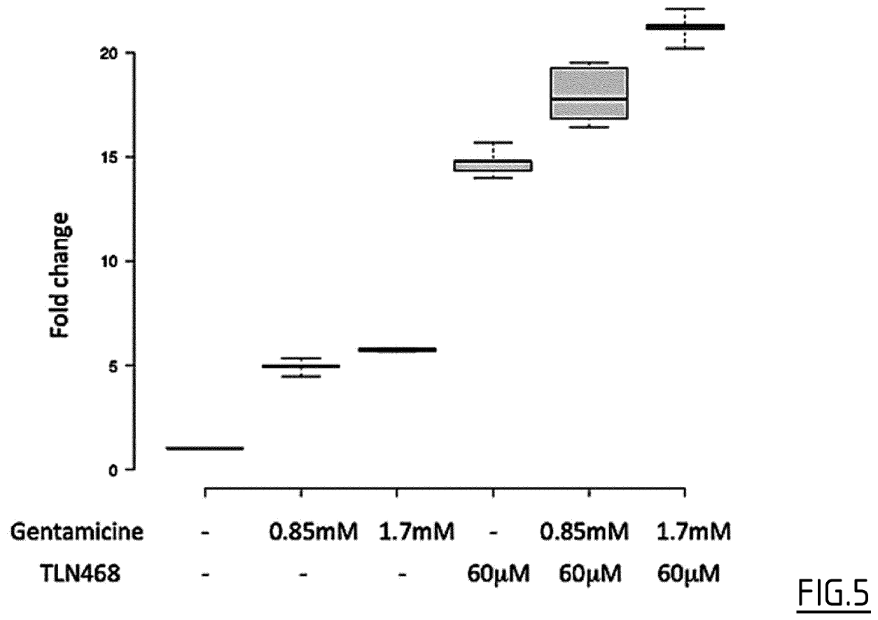

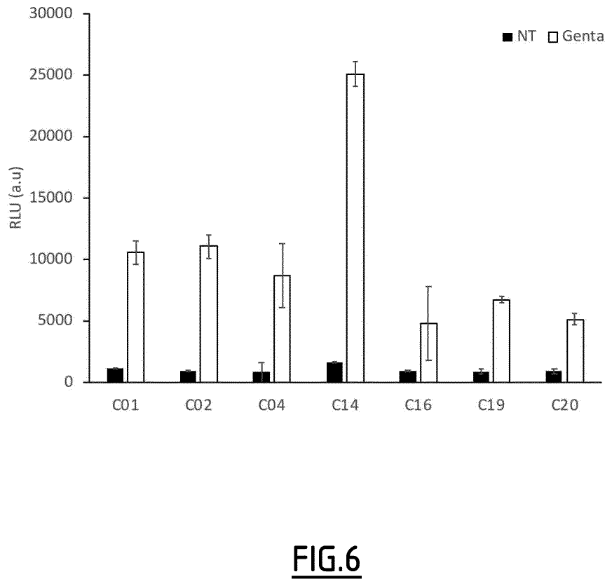

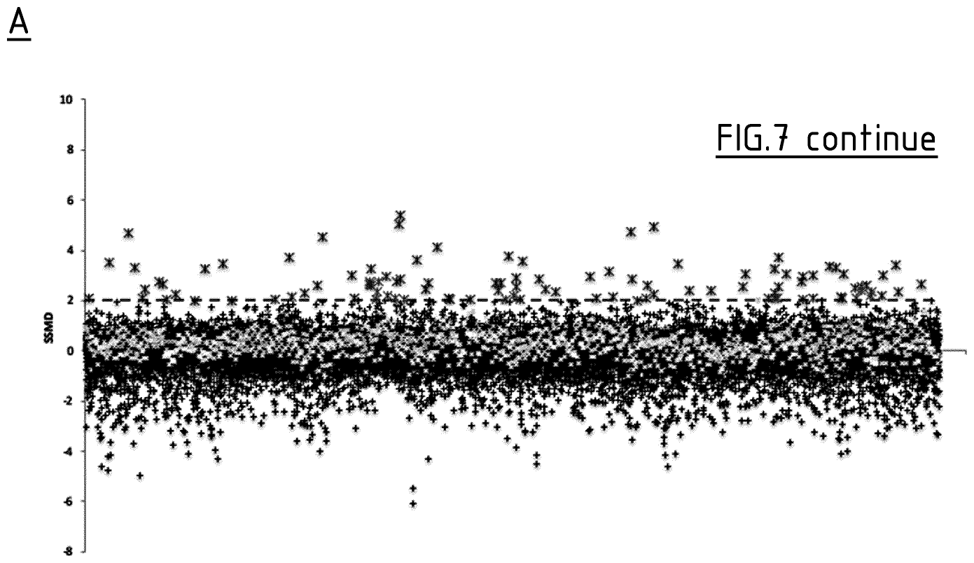

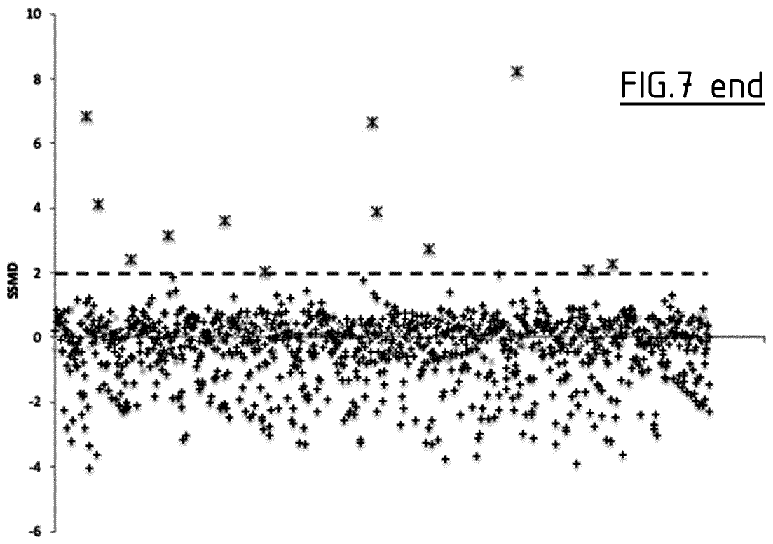

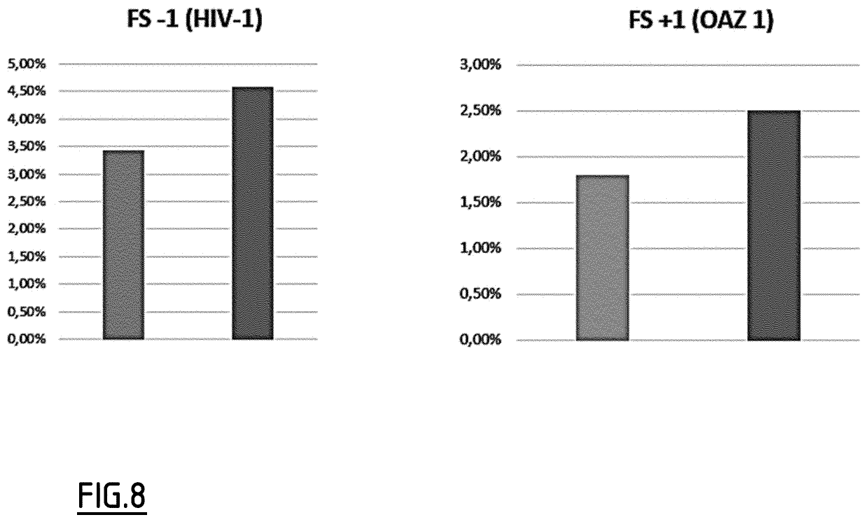

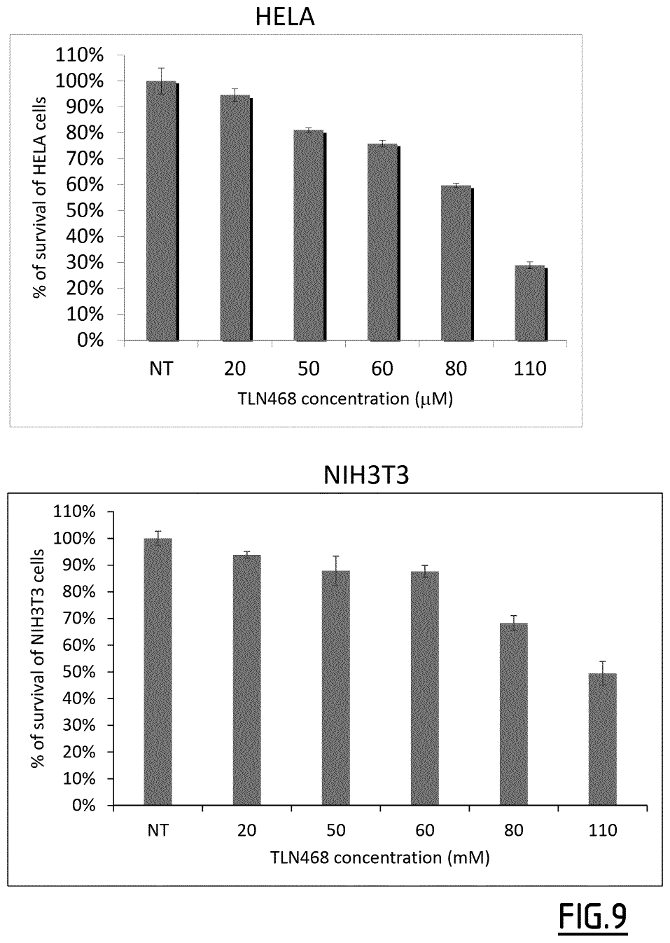

The inventors have screened 17,760 molecules (16,480 from ChemBridge library and 1,280 from Prestwick library) for their ability to efficiently stimulate stop codon readthrough. From this first screening they selected 465 molecules that display an increase factor greater or equal to 1.4 or a SSMD greater than or equal to 2 (FIG. 1.A). These first hits were then submitted to a second round of screening in the same conditions using duplicates for each molecule. The inventors retained 43 molecules for their ability to induce luciferase activity at least two fold. It is well-known that such screening can lead to the identification of a high number of false positive hits. To limit this and select only bona fide readthrough inducers, each of these 43 molecules were subjected to several independent assays to retain only the ones with a clear effect on stop codon readthrough.

Stop Codon Readthrough Quantification

Although highly sensitive and very convenient, the initial screening is subjected to several inherent biases. Indeed any molecule increasing the production of the Met-luciferase (mRNA transcription, stability, translation) or its secretion will lead to the identification of a false positive hit. To circumvent this the inventors checked the ability of each drug to stimulate readthrough in a dual reporter system to quantify stop codon readthrough efficiency (FIG. 1.B).

First this reporter system carries enzymatic activities (β-galactosidase and Firefly-luciferase) different from the one used in the initial screen. Second the β-galactosidase is used as an internal control to normalise expression level. So the use of this second reporter system eliminates many false positive hits potentially selected during the initial screen. For each tested molecule three independent measurements have been realised (FIG. 1.B). Only four molecules induce more than 2-fold change in PTC readthrough (data not shown). This second reporter system was very efficient to eliminate false positive hits, however it is still a reporter system using enzymatic activity. The inventors decided to use a more physiological system to test the last six potential hits.

Restoration of TP53 Protein Expression in HDQP-1 Cells

The inventors used the human cell line HDQ-P1 that carries the nonsense mutation R213X in its endogeneous TP53 gene. The expression of the full-length p53 is done by western-blot. The advantages of this third system are the use of an endogenous nonsense mutation and a direct visualisation of the final product induced by the potential hit (i.e. the full-length protein). Results are presented FIG. 2.A, and clearly reveal that two molecules stimulate the production of the full-length p53: TLN68 and TLN309. The level of induction is even higher to the one obtained in presence of G418 (one of the more efficient known readthrough inducers). Interestingly the inventors simultaneously observed an accumulation of the truncated form that could corresponds to the stabilisation of the TP53 mRNA through the inhibition of NMD. To confirm this they performed a RT-qPCR on the TP53-R213X mRNA and shown that TLN68 stabilises 6-fold this mRNA, whereas TLN309 shows a marginal stabilisation of the mRNA (FIG. 2.B).

A structural review of these two last candidates (FIG. 2.C) indicates that TLN309 is a 4-aminoquinoline similar to chloroquine that displays an autophagy-lysosomal inhibitory activity and promotes a ribosome biogenesis stress. The inventors decided to further pursue this analysis only with TLN68 that is a 2-guanidino quinazoline, named translectine (FIG. 2.C).

Functional TP53 Expression Restored by Stop Codon Readthrough

TLN68 is a very promising hit to restore the expression of the protein from a gene interrupted by a nonsense mutation. However restoring the expression of a full-length protein is a prerequisite to correct a defective gene, but this is not sufficient to warrant the functionality of the readthrough protein. Indeed, the inventors have shown that at least three tRNA (Tyr, Gln, Lys) can be used to readthrough UAG codon. Obviously the amino acid identity may strongly modify the activity of the restored full-length protein. So they tested with two different systems whether the full-length p53 expressed in presence of TLN68 is functional or not. First they used a plasmid carrying a luciferase gene under the control of a p53 promoter (FIG. 3.A), second they quantified by RT-qPCR the expression level of Bax mRNA that is one of the major targets of p53 (FIG. 3.B). The treatment by TLN68 induces a 3.5-fold increase of the p53-dependent luciferase, which is coherent with the 3-fold induction observed with bax mRNA level. This confirms that the readthrough p53 is at least partially active. Altogether these independent assays indicate that TLN68 is a promising readthrough inducer. During all these assays the inventors systematically used the same mutation (R213X). So, now that it is established that TLN68 induces stop codon readthrough on this premature stop codon, one may ask about its spectrum of action on various stop codons.

To the contrary, no protein is revealed in the presence of TLN236, suggesting that this drug does not act efficiently on stop codon readthrough.

Specificity of TLN68

To answer the question of TLN68 specificity the inventors selected the 40 more frequent premature nonsense mutations found in DMD gene responsible for Duchenne and Becker muscular dystrophy (Table 1; FIG. 4.A).

TABLE 1
Frequency of the 40 most frequent DMD
nonsense mutations

| | | Fre- | SEQ |
|---|---|---|---|
| | | quency | ID |
| Name | Nonsense mutation | (%) | NO: |
| R145X | AGC TGG GTC TGA CAA TCA ACT | 0.75 | 5 |
| S147X | GTC CGA CAA TGA ACT CGT AAT | 0.14 | 6 |
| Q194X | TCA GCC ACA TAA CGA CTG GAA | 2.63 | 7 |
| R195X | GCC ACA CAA TGA CTG GAA CAT | 0.61 | 8 |
| Q267X | GAA CAT TTT TAG TTA CAT CAT | 2.63 | 9 |
| R539X | TTG GGA GAT TGA TGG GCA AAC | 0.34 | 10 |
| Q555X | GTT CTT TTA TAA GAC ATC CTT | 0.07 | 11 |
| L654X | TGG GAT ATT TAA CAT CAA AAA | 0.07 | 12 |
| E761X | GAC TTA AAA TAA AAA GTC AAT | 0.55 | 13 |
| R768X | GCC ATA GAG TGA GAA AAA GCT | 0.82 | 14 |
| R1051X | AAT AAA CTC TGA AAA ATT CAG | 0.55 | 15 |
| W1075 | AAG GAG GAA TAG CCT GCC CTT | 2.63 | 16 |
| El182X | GAG TAT CTT TAG AGA GAT TTT | 0.07 | 17 |
| W1268X | TGG GCA TGT TGA CAT GAG TTA | 0.07 | 18 |
| R1577X | CGT AAG ATG TGA AAG GAA ATG | 0.68 | 19 |
| R1666X | GTC ACC TCC TGA GCA GAA GAG | 0.75 | 20 |
| R1844X | GAG AGA AAG TGA GAG GAA ATA | 0.27 | 21 |
| R1868X | AGG TCT CAA TGA AGA AAA AAG | 0.61 | 22 |
| W1879X | TCT CAT CAG TGA TAT CAG TAC | 0.07 | 23 |
| Y1882X | TGG TAT CAG TAA AAG AGG CAG | 0.14 | 24 |
| W1956X | AGC AAG CGC TAG CGG GAA ATT | 0.07 | 25 |
| R1967X | GCT CAG TTT TGA AGA CTC AAC | 0.61 | 26 |
| E2035X | TTT AAG CAA TAG GAG TCT CTG | 5.26 | 27 |
| R2095X | TAC AAG GAC TGA CAA GGG CGA | 0.55 | 28 |
| E2286X | ATA AGC CCA TAA GAG CAA GAT | 2.63 | 29 |
| Q2526X | AGG CGT CCC TAG TTG GAA GAA | 0.07 | 30 |
| R2553X | ATT ACG GAT TGA ATT GAA AGA | 0.82 | 31 |
| Q2574X | CGG AGG CAA TAG TTG AAT GAA | 2.63 | 32 |
| K2791X | GAA CTT CGG TAA AAG TCT CTC | 2.63 | 33 |
| R2870X | GAG ACT GTA TGA ATA TTT CTG | 0.82 | 34 |
| R2905X | CGG CTT CTA TGA AAG CAG GCT | 0.34 | 35 |
| W2925X | TCC GCT GAC TGA CAG AGA AAA | 0.14 | 36 |
| R2982X | AAG GCA CTT TGA GAA GAA AAT | 0.55 | 37 |
| R3034X | GTC GAG GAC TGA GTC GTC CAG | 0.55 | 38 |
| S3127X | TTG AGC CTG TGA GCT GCA TGT | 2.63 | 39 |
| R3190X | GAT ACG GGA TGA ACA GGG AGG | 0.89 | 40 |
| R3345X | TTT TCT GGT TGA GTT GCA AAA | 0.48 | 41 |
| R3370X | GAA GAT GTT TGA GAC TTT GCC | 1.02 | 42 |
| R3381X | AAC AAA TTT TGA ACC AAA AGG | 1.57 | 43 |
| R3391X | AAG CAT CCC TGA ATG GGC TAC | 1.5 | 44 |

Each nonsense mutation (+9 nucleotides in each side) has been cloned into the dual reporter previously used. Stop codon readthrough efficiency has been quantified either without drug or in presence of gentamicin or TLN68, in 6 independent experiments. The results shown FIG. 4.B indicate that TLN68 promotes stop codon readthrough not only onto a large variety of sequences (28 out of 40 tested) but also outperforms gentamicin on this set of sequences. The inventors conclude TLN68 is active onto a broad variety of sequences, although its action is sequence-dependent as most of the known readthrough inducers.

TLN68 has an Additive Effect with Gentamicin to Induce Readthrough

The inventors then test effect of a combined treatment of TLN68 with one of the most used readthrough molecule, gentamicin. NIH3T3 cells were transfected with dual reporter vector harboring R213X mutation and treated 24 h with each drug separately or together. Gentamicin and TLN68 induce a readthrough level of 10 and 11% respectively and the combination of the two drugs allow a readthrough level of 21% suggesting an additive effect between these molecules (FIG. 5).

TLN68 Effect on Translation is Specific of Readthrough Events

To determinate if TLN68 alter global translational fidelity or if it is specific of readthrough event the inventors tested capacity of this molecule to induce frameshifting. They used a frameshift target which have already been used to study the minus1 frameshift signal of the retrovirus HIV-1 (Stahl et al, 1995). This 54 nucleotide sequence was inserted in the dual reporter vector and used to transfect NIH3T3 cells. Transfected cells were treated or not with TLN68 at 80 μM. Results demonstrate that TLN68 has no impact on translational frameshifting suggesting that this molecule does not have an overall impact on translation fidelity but might rather act specifically on readthrough (FIG. 8).

TLN68 has Moderate Toxicity on Mammalian Cell Lines

The inventors tested the effect of TLN68 on viability of NIH3T3, HDQ-P1 and HeLa cell lines by using a tetrazolium salt which is cleaved only by metabolically active cells. Cells were treated or not with a range of TLN68 doses during 24 h and viability was measured. FIG. 9 shows that cell viability slowly decreases as the TLN68 concentration increases to reach 80% of viable cells for NIH3T3 which are the most resistant cell line cells and 70% for HDQ-P1 cells at the maximal dose used.

### Example 3: Tests of the Ability of Different Compounds According to the Invention to Induce PTC Readthrough

Protocol:

Different compounds of the invention have been tested for their ability to induce readthrough on the nonsense mutation W1268X or R213X in HeLa cells.

To this aim the inventors used the pAC99 dual reporter plasmid. This dual reporter is used to quantify stop-codon readthrough through the measurement of luciferase and beta-galactosidase (internal normalisation) activities. Readthrough levels for nonsense mutations were analysed in the presence or absence of tested molecules. Cells are seeded in a 6-well plate. The next day, cells are transfected with the reporter plasmid using the JetPei reagent (Invitrogen). The following day, they are rinsed with fresh medium, with or without readthrough inducers. Cells were harvested 24 hours later, with trypsin—EDTA (Invitrogen), lysed with Glo lysis buffer (Promega) and beta-galactosidase and luciferase activities were assayed as previously described. Readthrough efficiency was estimated by calculating the ratio of luciferase activity to beta-galactosidase activity obtained with the test construct, with normalisation against an in-frame control construct. At least five independent transfection experiments were performed for each assay.

Results:

The results are as follows:

| | | Inducting factor of | Inducting factor of |
|---|---|---|---|
| | | readthrough as | readthrough as |
| | | compared to | compared to |
| | | untreated cells | untreated cells |
| | IC50 in | (measured on | (measured on |
| | HeLa cells | nonsense mutation | nonsense mutation |
| Molecule | (in μM) | W1268X) | R213X) |
| TLN68 | | 9 | 14 |
| EC-18 | 60 | 69 | 50 |
| EC-141 | 40 | 16 | — |
| EC-85 | 20 | 15 | 16 |
| EC-11 | 40 | 11 | — |
| EC-35 | 20 | 11 | 14 |
| EC-30 | 80 | 10 | — |
| EC-265 | 60 | 19 | 40 |
| EC-288 | 20 | 68 | 55 |
| EC-130 | 10 | 21 | 33 |
| EC-335 | 10 | 33 | 38 |

As a conclusion, the compounds of the invention are PTC readthrough promoters which are very efficient.

### Example 4: Ribosome Profiling Experiment Using TLN68

Objective

It is important to demonstrate that TLN68 acts specifically on premature termination codons (PTC) and has no effect on natural stop codons. To answer this question, the inventors performed a Ribosome profiling (RiboSeq) experiment that allows genome-wide mapping of all ribosome footprints. They tested three different conditions: untreated cells, 0.5 mg/ml G418, 80 mM TLN468 for 24 h. Each condition has been performed in triplicate for statistical reasons.

Ribosome Profiling Experiments

HeLa cells were plated at JO at 1 million cells per plate with 10 ml of MEM supplemented with 10% fœtal Calf Bovine Serum, 1% glutamine, non-essential amino acids and antibiotics-antimycotic (Gibco). At J+1, 80 mM TLN468 is added. At J+2, cells are collected. The medium was removed and then directly laid on liquid nitrogen bath, put to −70° C. before being scratched. The cells were collected with addition of Polysome Extraction Buffer (10mMTris CH₃COONa pH7.6; 10 mM (CH₃COO)₂Mg; 10 mM NH₄Cl; 1% Triton; 2 mM DTT).

Polysomes were extracted with the addition of 2× complete EDTA-free protease inhibitor Roche and 1U Rnase Inhibitor Murine (Biolabs). The Ribosome Protected Fragments (RPF) were generated by 1 h of digestion with 15 U Rnase I (Ambion)/OD260ₙₘ at 25° C.). Monosomes are separated on 24% sucrose cushion at +4° C. then treated by DNasel. RNA is extracted by phenol at 65° C., CHCL₃ and precipitated by ethanol in 0.3M CH₃COONa pH5.2 then loaded on a 17% acrylamide-bisacrylamide (19:1) gel with 7 M urea and 1× Tris-acetate-EDTA (TAE). RPF at 28- to 34-nt are excised from gel and precipitated in ethanol in presence of glycogen. RPF are depleted from ribosomal RNA with the Ribo-zero Human kit (Illumina) following the manufacturer's recommendations. The RPF libraries are made using the Transcriptome TruSeq modified kit and sequenced on NextSeq 500 High Single Read 75 bases.

Results

Data are analyzed by homemade scripts. Cells are flash-frozen in liquid nitrogen and polysomes extracted. Once the data obtained, the inventors informatically removed all contaminating rRNA fragments and used a homemade docker package (RiboDoc) to map reads on the human transcriptome using the human genome (hg38) as reference. They obtained a total of 122 497 388, 160 481 925 and 51 860 606 uniquely mapped reads for untreated, G418 and TLN68 respectively. They first checked the ribosomal footprints length distribution. The results show that the majority of footprints are 30 nucleotides (30nts) long for the untreated cells (Hela) and cells treated with G418, whereas the footprints are 29 nucleotides (29nts) long for cells treated with TLN68. This length is perfectly what is expected for a ribosome footprint that must be comprised between 28-31 nucleotides. The observed difference between TLN68 and the other samples probably simply reflects variations during RPF preparation, without biological meaning.

The inventors selected only the 29nts and 30nts long ribosome footprints for TLN68 and HeLa/G418 samples respectively to perform a metagene analysis in which all annotated CDS are pooled and the number of ribosome footprint 5′ ends at each nucleotide position is determined. This step allows confirming that the analysis is performed on actively translating ribosomes. Indeed, the inventors can clearly observe a signal with a periodicity of 3 (data not shown). This periodicity represents ribosomes moving codon by codon along the mRNA. The inventors classically observe a peak at the start and stop codons because initiation and termination steps are kinetically slower than the elongation steps, promoting the accumulation of ribosomes at the start and stop codons. They can also observe weak signals upstream the start codon in the 5′UTR, which could correspond to uORFs. Immediately downstream of the stop codon the translation signal disappears, except in samples treated with G418. This signal represents genome-wide stop codon readthrough promoted by G418. The signal quickly disappears because readthrough efficiency is low and ribosomes quickly encounters a second stop (the median distance between the normal stop codon and the next in-frame codon is 18 codons for the human genome). Interestingly the inventors did not observe any genome-wide readthrough for the untreated cells and cells treated with TLN68.

This indicates that TLN68 specifically targets premature stop codons and has no effect on natural terminating codons.

The same experiment has been performed using other compounds of formula (I) or (II) or one of their salts of the invention, and preferably compound EC-18 or EC-288, instead of TLN68.

1. A method for preventing and/or treating a disease caused by a nonsense mutation, said disease being chosen from genetic diseases caused by a nonsense mutation and cancers caused by a nonsense mutation which is present in a tumor-suppressor gene, in a subject, which comprises administering to said subject at least one compound of formula (I), or one of its pharmaceutically acceptable salts: 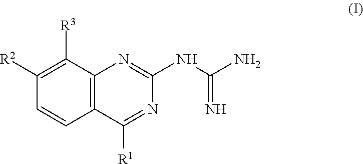
wherein:
R1 is a C1-C6 alkyl radical;
R2 is H, a halogen atom or a C1-C6 alkoxy radical; and
R3 is H, a C1-C6 alkyl radical or a C1-C6 alkoxy radical.

2. The method according to claim 1, wherein the genetic disease or the cancer is caused by the presence of a nonsense mutation in a coding sequence of interest.

3. The method according to claim 1, wherein R2 and R3 are each not simultaneously H.

4. The method according to claim 1, wherein:
R1 is methyl or ethyl;
R2 is H, a halogen atom or a methoxy radical; and
R3 is H, a methyl radical or a methoxy radical.

5. The method according to claim 1, wherein:
R1 is methyl or ethyl;
R2 a halogen atom or
a methoxy radical; and
R3 is H or a methyl radical.

6. The method according to claim 1, wherein the compound is chosen from the following compounds and their pharmaceutically acceptable salts:
TLN68, which is 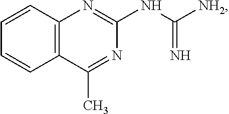
EC-18, which is 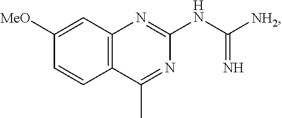 [SMILES: COc1ccc2c(C)nc(NC(=N)N)nc2c1]
EC-35 which is 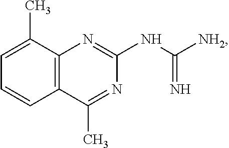 [SMILES: Cc1nc(NC(=N)N)nc2c(C)cccc12]
EC-30 which is 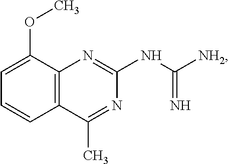 [SMILES: COc1cccc2c(C)nc(NC(=N)N)nc12]
EC-141 which is 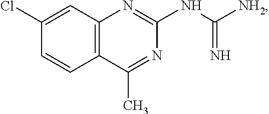 [SMILES: Cc1nc(NC(=N)N)nc2cc(Cl)ccc12] 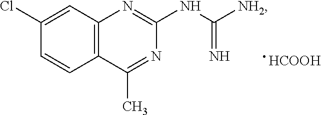 [SMILES: Cc1nc(NC(=N)N)nc2cc(Cl)ccc12 ; "· HCOOH"]
EC-85 which is 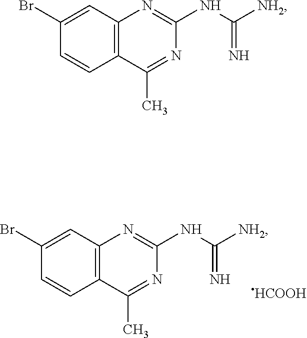 [SMILES: Cc1nc(NC(=N)N)nc2cc(Br)ccc12 ; Cc1nc(NC(=N)N)nc2cc(Br)ccc12 ; "·HCOOH"]
EC-11 which is 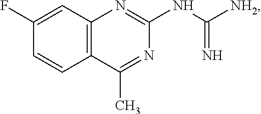 [SMILES: Cc1nc(NC(=N)N)nc2cc(F)ccc12]
EC-288 which is 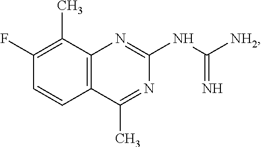 [SMILES: Cc1nc(NC(=N)N)nc2c(C)c(F)ccc12]
EC-130 which is 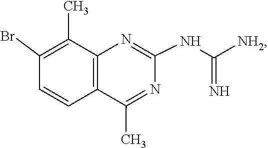 [SMILES: Cc1nc(NC(=N)N)nc2c(C)c(Br)ccc12]
EC-335 which is 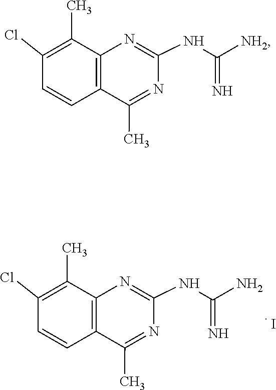
EC-265 which is

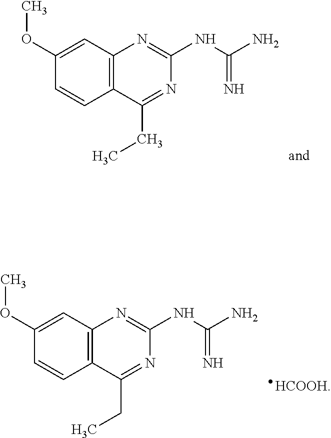 [SMILES: CCc1nc(NC(=N)N)nc2cc(OC)ccc12 ; CCc1nc(NC(=N)N)nc2cc(OC)ccc12 ; "· HCOOH."]

7. The method according to claim 1, wherein the compound is chosen from the following compounds and their pharmaceutically acceptable salts:
EC-18, which is 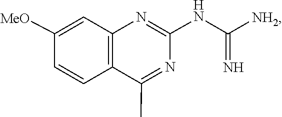 [SMILES: COc1ccc2c(C)nc(NC(=N)N)nc2c1]
EC-141 which is 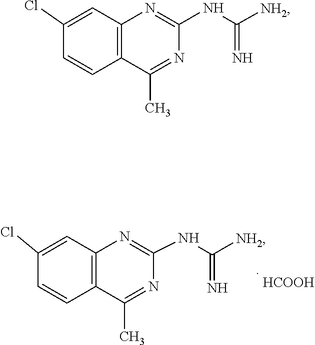 [SMILES: Cc1nc(NC(=N)N)nc2cc(Cl)ccc12 ; Cc1nc(NC(=N)N)nc2cc(Cl)ccc12 ; "HCOOH"]
EC-85 which is 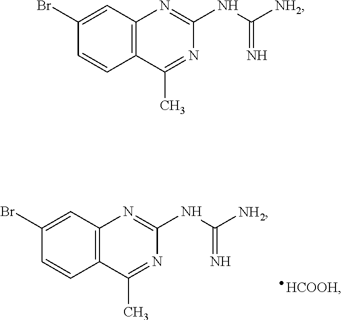 [SMILES: Cc1nc(NC(=N)N)nc2cc(Br)ccc12 ; Cc1nc(NC(=N)N)nc2cc(Br)ccc12]
EC-11 which is 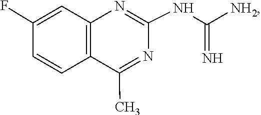 [SMILES: Cc1nc(NC(=N)N)nc2cc(F)ccc12]
EC-288 which is 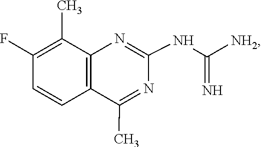 [SMILES: Cc1nc(NC(=N)N)nc2c(C)c(F)ccc12]
EC-130 which is 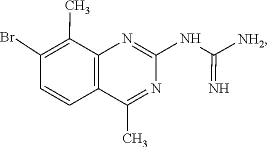 [SMILES: Cc1nc(NC(=N)N)nc2c(C)c(Br)ccc12]
EC-335 which is 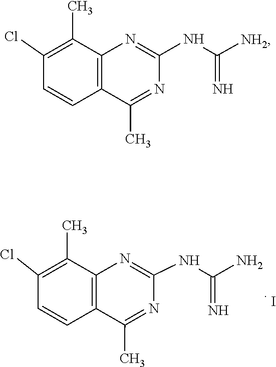
EC-265 which is

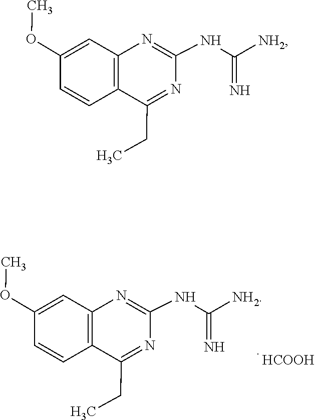 [SMILES: CCc1nc(NC(=N)N)nc2cc(OC)ccc12 ; CCc1nc(NC(=N)N)nc2cc(OC)ccc12 ; "HCOOH"]

8. The method according to claim 1, wherein the genetic disease caused by a nonsense mutation is chosen from cystic fibrosis due to a nonsense mutation G542X in the cystic fibrosis transmembrane conductance regulator gene, Duchenne muscular dystrophy due to a nonsense mutation in dystrophin, beta thalassemia due to nonsense mutation in β-globin, Niemann-Pick disease type A, B or C due to nonsense L261X mutation in acid sphingomyelinase, Hurler syndrome, Dravet syndrome, spinal muscular atrophy, myoadenylate deaminase deficiency, antithrombin III deficiency, alpha-1 antitrypsin deficiency, apolipoprotein deficiency of apolipoprotein Al, B, CII or E, adenine phosphoribosyltransferase (APRT) deficiency, haemophilia A due to nonsense mutation in Factor VIII), haemophilia B due to nonsense mutation in Factor IX), Von Willebrand disease due to a nonsense mutation in Von Willebrand factor, Fanconi anemia-group C, Marfan syndrome, Gaucher disease, Donohue syndrome, oculocerebrorenal syndrome of Lowe and Xeroderma pigmentosum.

9. Compound of formula (II) or one of its pharmaceutically acceptable salts: 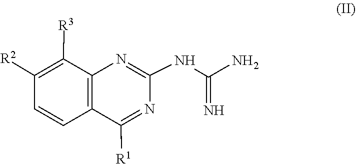
wherein:
R1 is a C1-C6 alkyl radical, preferably methyl or ethyl;
R2 is a halogen atom or a C1-C6 alkoxy radical, preferably methoxy; and
R3 is H or a C1-C6 alkyl radical, preferably methyl,
with the proviso that when R2 is a C1-C6 alkoxy radical, then R1 is ethyl, and
when R2 is a halogen and R3 is H then the compound is in the form of a pharmaceutically acceptable salt such as a salt with formic acid or with iodine.

10. Compound according to claim 9, wherein it is chosen from the following compounds and their pharmaceutically acceptable salts:
EC-141 which is 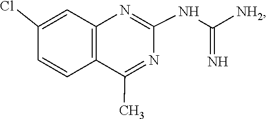 [SMILES: Cc1nc(NC(=N)N)nc2cc(Cl)ccc12]
EC-85 which is 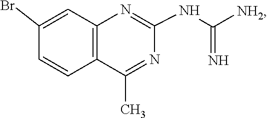 [SMILES: Cc1nc(NC(=N)N)nc2cc(Br)ccc12]
EC-288 which is 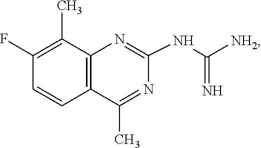 [SMILES: Cc1nc(NC(=N)N)nc2c(C)c(F)ccc12]
EC-130 which is 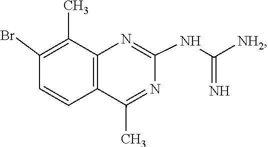 [SMILES: Cc1nc(NC(=N)N)nc2c(C)c(Br)ccc12]
EC-335 which is 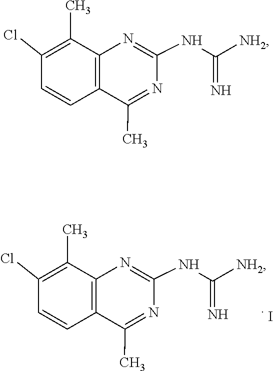
EC-265 which is

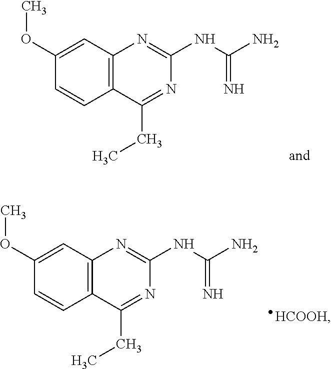 [SMILES: CCc1nc(NC(=N)N)nc2cc(OC)ccc12 ; CCc1nc(NC(=N)N)nc2cc(OC)ccc12]

11. A method for treating a disease, which comprises administering at least one compound according to claim 9.

12. Composition comprising, in a pharmaceutically acceptable carrier, at least one compound according to claim 9 or one of its pharmaceutically acceptable salts.

13. A method for treating a disease caused by a nonsense mutation, said disease being chosen from genetic diseases caused by a nonsense mutation and cancers caused by a nonsense mutation which is present in a tumor-suppressor gene, in a subject, which comprises administering to said subject at least one product comprising:
a) a compound according to claim 1, or one of its pharmaceutically acceptable salts, and
b) at least one other drug,
said compound and said at least one other drug being formulated in the product for a simultaneous, separate or sequential administration.

14. A method for treating cancer, and/or for preventing cancer metastasis, and/or for preventing cancer recurrence, and/or for decreasing resistance to a chemotherapeutic drug, in a subject, comprising administering to said subject at least one product comprising:
a) a compound according to claim 9, or one of its pharmaceutically acceptable salts, and
b) at least one chemotherapeutic drug,
said compound and said at least one chemotherapeutic drug being formulated in the product for a simultaneous, separate or sequential administration.

15. The method according to claim 13, or wherein the drug is chosen from ataluren, gentamicin, negamycin, clitocine, escin and a nonsense-mediated mRNA decay (NMD) inhibitors.

16. The method according to claim 14, wherein the drug is chosen from ataluren, gentamicin, negamycin, clitocine, escin and a nonsense-mediated mRNA decay (NMD) inhibitor.


SEQUENCE LISTING (44 sequences)

SEQ ID NO: 1 - DNA, 19 bp, Artificial Sequence
  Synthetic primer p53 forward
ccgcagtcagatcctagcg

SEQ ID NO: 2 - DNA, 21 bp, Artificial Sequence
  Synthetic primer p53 reverse
ccattgcttgggacggcaagg

SEQ ID NO: 3 - DNA, 20 bp, Artificial Sequence
  Synthetic primer Bax forward
gctgttgggctggatccaag

SEQ ID NO: 4 - DNA, 20 bp, Artificial Sequence
  Synthetic primer Bax reverse
tcagcccatcttcttccaga

SEQ ID NO: 5 - DNA, 21 bp, Homo sapiens
agctgggtctgacaatcaact

SEQ ID NO: 6 - DNA, 21 bp, Homo sapiens
gtccgacaatgaactcgtaat

SEQ ID NO: 7 - DNA, 21 bp, Homo sapiens
tcagccacataacgactggaa

SEQ ID NO: 8 - DNA, 21 bp, Homo sapiens
gccacacaatgactggaacat

SEQ ID NO: 9 - DNA, 21 bp, Homo sapiens
gaacatttttagttacatcat

SEQ ID NO: 10 - DNA, 21 bp, Homo sapiens
ttgggagattgatgggcaaac

SEQ ID NO: 11 - DNA, 21 bp, Homo sapiens
gttcttttataagacatcctt

SEQ ID NO: 12 - DNA, 21 bp, Homo sapiens
tgggatatttaacatcaaaaa

SEQ ID NO: 13 - DNA, 21 bp, Homo sapiens
gacttaaaataaaaagtcaat

SEQ ID NO: 14 - DNA, 21 bp, Homo sapiens
gccatagagtgagaaaaagct

SEQ ID NO: 15 - DNA, 21 bp, Homo sapiens
aataaactctgaaaaattcag

SEQ ID NO: 16 - DNA, 21 bp, Homo sapiens
aaggaggaatagcctgccctt

SEQ ID NO: 17 - DNA, 21 bp, Homo sapiens
gagtatctttagagagatttt

SEQ ID NO: 18 - DNA, 21 bp, Homo sapiens
tgggcatgttgacatgagtta

SEQ ID NO: 19 - DNA, 21 bp, Homo sapiens
cgtaagatgtgaaaggaaatg

SEQ ID NO: 20 - DNA, 21 bp, Homo sapiens
gtcacctcctgagcagaagag

SEQ ID NO: 21 - DNA, 21 bp, Homo sapiens
gagagaaagtgagaggaaata

SEQ ID NO: 22 - DNA, 21 bp, Homo sapiens
aggtctcaatgaagaaaaaag

SEQ ID NO: 23 - DNA, 21 bp, Homo sapiens
tctcatcagtgatatcagtac

SEQ ID NO: 24 - DNA, 21 bp, Homo sapiens
tggtatcagtaaaagaggcag

SEQ ID NO: 25 - DNA, 21 bp, Homo sapiens
agcaagcgctagcgggaaatt

SEQ ID NO: 26 - DNA, 21 bp, Homo sapiens
gctcagttttgaagactcaac

SEQ ID NO: 27 - DNA, 21 bp, Homo sapiens
tttaagcaataggagtctctg

SEQ ID NO: 28 - DNA, 21 bp, Homo sapiens
tacaaggactgacaagggcga

SEQ ID NO: 29 - DNA, 21 bp, Homo sapiens
ataagcccataagagcaagat

SEQ ID NO: 30 - DNA, 21 bp, Homo sapiens
aggcgtccctagttggaagaa

SEQ ID NO: 31 - DNA, 21 bp, Homo sapiens
attacggattgaattgaaaga

SEQ ID NO: 32 - DNA, 21 bp, Homo sapiens
cggaggcaatagttgaatgaa

SEQ ID NO: 33 - DNA, 21 bp, Homo sapiens
gaacttcggtaaaagtctctc

SEQ ID NO: 34 - DNA, 21 bp, Homo sapiens
gagactgtatgaatatttctg

SEQ ID NO: 35 - DNA, 21 bp, Homo sapiens
cggcttctatgaaagcaggct

SEQ ID NO: 36 - DNA, 21 bp, Homo sapiens
tccgctgactgacagagaaaa

SEQ ID NO: 37 - DNA, 21 bp, Homo sapiens
aaggcactttgagaagaaaat

SEQ ID NO: 38 - DNA, 21 bp, Homo sapiens
gtcgaggactgagtcgtccag

SEQ ID NO: 39 - DNA, 21 bp, Homo sapiens
ttgagcctgtgagctgcatgt

SEQ ID NO: 40 - DNA, 21 bp, Homo sapiens
gatacgggatgaacagggagg

SEQ ID NO: 41 - DNA, 21 bp, Homo sapiens
ttttctggttgagttgcaaaa

SEQ ID NO: 42 - DNA, 21 bp, Homo sapiens
gaagatgtttgagactttgcc

SEQ ID NO: 43 - DNA, 21 bp, Homo sapiens
aacaaattttgaaccaaaagg

SEQ ID NO: 44 - DNA, 21 bp, Homo sapiens
aagcatccctgaatgggctac